(19) United States
(12) Patent Application Publication
(10) Pub. No.: US 2012/0003731 A1
(43) Pub. Date: Jan. 5, 2012

(54) ANALYZER AND METHOD FOR WASHING DISPENSER PROBE

(75) Inventor: Akihisa Kuroda, Shizuoka (JP)

(73) Assignee: Beckman Coulter, Inc., Brea (CA)

(21) Appl. No.: 13/256,020

(22) PCT Filed: Mar. 9, 2010
(86) PCT No.: PCT/JP2010/053883

§ 371 Date: Sep. 12, 2011

(30) Foreign Application Priority Data
Mar. 13, 2009 (JP) 2009-061733

(51) Int. Cl. C12M 1/34; B08B 7/04

(52) U.S. Cl. 435/288.7; 134/18

(57) ABSTRACT

The analyzer (1) of the present invention comprises: a reading section (23) for storing or obtaining specimen information including a sample type of a specimen, and specimen container information; a liquid level detecting section (51) for detecting a liquid level and/or an interface of the specimen; a dispensing apparatus (5) for dispensing a specimen; a washing apparatus (6) for washing a dispensing probe (50); a calculating section (202) for calculating a contamination adhering range of an external wall surface of the dispensing probe (50), based on the sample type, specimen suction position and specimen container information stored or obtained by the reading section (23), as well as liquid level and/or interface information of the specimen detected by the liquid level detecting section (51); and a washing control section (200) for controlling a washing range based on the contamination adhering range.

### TECHNICAL FIELD

The present invention relates to an analyzer for analyzing a blood sample and a method for washing a dispensing probe.

### BACKGROUND ART

Conventionally, blood sugar and hemoglobin Alc (HbAlc) have been used as diagnostic markers of diabetes. In automatic analyzers, a blood sample drawn from a subject is separated into blood plasma and blood cells by means of centrifugation to analyze blood sugar from the blood plasma sample, and hemoglobin Alc from the blood cell sample. Alternatively, analysis is performed with specimens dispensed from whole blood in order to analyze a red blood cell component, hemoglobin Alc. For the dispensing and analyzing of a specimen, which is separated into layers of blood plasma and blood cells, or a whole blood specimen, together with a blood plasma (blood serum) specimen or the like using one automatic analyzer, an important problem to be solved is to prevent carryover to other specimens. To that end, an automatic analyzer is proposed, which is capable of selecting washing ranges of a dispensing pipette for two stages, based on a contaminant range of the dispensing pipette during the dispensation (see, for example, Patent Literature 1).

### CITATION LIST

### Patent Literature

Patent Literature 1: Japanese Laid-Open Publication No. 2002-40035

### DISCLOSURE OF THE INVENTION

### Summary of Invention

### Technical Problem

In the analyzer disclosed in Patent Literature 1, the length of a sample diluting pipette inserted into a specimen in a sample container is different in the case where the sample diluting pipette sucks blood plasma from a specimen, which is separated into layers of blood plasma and blood cells, from the case where the sample diluting pipette sucks out the blood cells. Thus, the adhesion ranges of blood plasma and specimens adhered to an external wall of the sample diluting pipette are also different from one another. Accordingly, the analyzer is configured to be able to select and control a washing range of the sample diluting pipette after the suction of blood plasma as well as a washing range of the sample diluting pipette after the suction of blood cells, though the analyzer can only select the washing range based on the contamination range. Even if the type of adhered samples is different, such as if blood plasma is adhered in a wide range, if whole blood is adhered in a wide range, or if a specimen, which is separated into layers of blood plasma and blood cells, is adhered in a wide range, the sample diluting pipette will be washed under the same washing conditions. Under the washing conditions for handling the most severe contamination, the extended washing time and consumption of a large amount of washing water become problematic.

The present invention is intended to solve such a problem as described above. It is an objective of the present invention to provide: an analyzer and a method for washing a dispensing probe, capable of controlling washing conditions of a dispensing probe, based on the type of contamination or the like adhered to the dispensing probe, to shorten the total washing time and reduce the amount of washing water to be used.

### Solution to Problem

An analyzer according to the present invention is capable of analyzing different samples as a specimen, the analyzer optically analyzing a reactant of the specimen and a reagent, characterized in comprising: an analysis information obtaining section for storing or obtaining specimen information including a sample type of the specimen and a specimen suction position, and specimen container information; a liquid level estimating section for estimating a liquid level and/or an interface of the specimen; a dispensing section for dispensing the specimen using a dispensing probe; a washing section for washing the dispensing probe; a calculating section for calculating a contamination adhering range of an external wall surface of the dispensing probe, based on the sample type, the specimen suction position, and the specimen container information stored or obtained by the analysis information obtaining section as well as liquid level and/or interface information of the specimen estimated by the liquid level estimating section; and a washing control section for controlling a washing range of the dispensing probe, based on the contamination adhering range of the external wall surface of the dispensing probe calculated by the calculating section, thereby achieving the objective described above.

Further, an analyzer according to the present invention is capable of analyzing different samples as a specimen, the analyzer optically analyzing a reactant of the specimen and a reagent, characterized in comprising: an analysis information obtaining section for storing or obtaining specimen information including a sample type of the specimen and a specimen suction position; a liquid level detecting section for detecting a liquid level and/or an interface of the specimen; a dispensing section for dispensing the specimen using a dispensing probe; a washing section for washing the dispensing probe; a calculating section for calculating a contamination adhering range of an external wall surface of the dispensing probe, based on the sample type, the specimen suction position, and the specimen container information stored or obtained by the analysis information obtaining section as well as liquid level and/or interface information of the specimen detected by the liquid level detecting section; and a washing control section for controlling a washing range of the dispensing probe, based on the contamination adhering range of the external wall surface of the dispensing probe calculated by the calculating section.

In the invention described above, the analyzer according to the present invention is characterized in that the washing control section controls a lowering speed of the dispensing probe into the washing section based on the sample type of the specimen.

In the invention described above, the analyzer according to the present invention is characterized in that the specimen information includes a sample type, sample viscosity, a specimen dispensing amount, a specimen suction position and an analysis menu information.

In the invention described above, the analyzer according to the present invention is characterized in that the washing control section controls the lowering speed of the dispensing probe in accordance with a viscosity category of the specimen.

In the invention described above, the analyzer according to the present invention is characterized in that the washing section comprises: a washing vessel having an opening at an upper part thereof, the opening allowing the dispensing probe to be inserted therein; a spout washing water supplying section for spouting washing water in a region of the upper part of the washing vessel; and a washing water controlling section for controlling the amount of the washing water spouted from the spout washing water supplying section based on the sample type of the specimen to be washed.

In the invention described above, the analyzer according to the present invention is characterized in that the washing section comprises: a reservoir vessel for allowing overflow the washing water, the reservoir vessel having an opening at an upper part thereof for allowing the dispensing probe to be inserted therein; an overflow vessel for discharging the overflow washing water from the reservoir vessel; a reserve washing water supplying section for supplying the washing water to the reservoir vessel; and a washing water controlling section for controlling the amount of the washing water supplied from the reserve washing water supplying section based on the sample type of the specimen to be washed.

In the invention described above, the analyzer according to the present invention is characterized in that the washing water controlling section controls at least the reservoir vessel to be in an overflow state before an extrusion liquid, which is discharged from the dispensing probe to wash an internal wall surface thereof at an upper part of the reservoir vessel, falls and reaches the washing water reserved in the reservoir vessel.

In the invention described above, the analyzer according to the present invention is characterized in that the washing water controlling section controls at least the reservoir vessel to be in an overflow state before a tip of the dispensing probe is dipped in the washing water reserved in the reservoir vessel by the lowering of the dispensing probe into the washing section.

In the invention described above, the analyzer according to the present invention is characterized in that an opening of the overflow vessel is formed to have an inclined surface, which inclines downwardly from the opening of the reservoir vessel.

In the invention described above, the analyzer according to the present invention is characterized in that the washing water controlling section performs a control to allow the washing water reserved in the reservoir vessel to overflow before the extrusion liquid discharged from the dispensing probe for washing the internal wall surface thereof falls into and reach the washing water, and after the discharging of the extrusion liquid for washing the internal wall surface from the dispensing probe ends, the washing water controlling section performs a control to allow the washing water reserved in the reservoir vessel to stop overflowing after the discharged extrusion liquid falls and reaches the washing water.

In the invention described above, the analyzer according to the present invention is characterized in that the washing water controlling section performs a control to resume the overflow before the dispensing probe is lowered in the washing section and the tip of the dispensing probe is dipped in the washing water reserved in the reservoir vessel, and the washing water controlling section performs a control to stop the overflow before the dispensing probe is pulled up from the reservoir vessel.

In the invention described above, the analyzer according to the present invention is characterized in that the analyzer comprises a spout washing water supplying section for spouting washing water in an upper part region of the washing vessel.

In the invention described above, the analyzer according to the present invention is characterized in that the washing water controlling section controls the washing water spouted from the spout washing water supplying section to allow at least the reservoir vessel to be in an overflow state when the washing water falls into the reservoir vessel.

In the invention described above, the analyzer according to the present invention is characterized in that the washing control section selects, for performing washing, washing by washing-water spouting by the spout washing water supplying section and/or dip-washing with the washing water reserved in the reservoir vessel, in accordance with the sample type dispensed by the dispensing probe.

In the invention described above, the analyzer according to the present invention is characterized in that the calculating section calculates a specimen contamination range of each layer of the external wall surface of the dispensing probe, based on a suction position of each layer of the specimen which is separated into layers, and the specimen container information, stored or obtained by the analysis information obtaining section, as well as liquid level position information of each layer of the specimen which is separated into layers, detected by the liquid level detecting section; the washing control section controls an insertion distance or the lowering speed of the dispensing probe to the washing section in accordance with the specimen contamination range of each layer calculated by the calculating section; and/or the washing water controlling section controls the amount of the washing water.

In the invention described above, the analyzer according to the present invention is characterized in that the liquid level detecting section comprises: a liquid level detecting part for detecting a liquid level from the change in capacitance between the dispensing probe and a metal plate disposed in the periphery of a specimen container; and an interface detecting section for detecting a pressure during ascent or descent while performing suction of the dispensing probe in the specimen using a pressure detecting section within the dispensing probe, to detect an interface of the specimen which is separated into layers in a specimen container, based on the detected pressure change.

In the invention described above, the analyzer according to the present invention is characterized in that the sample type of the specimen is blood plasma, blood serum, whole blood or a specimen which is separated into layers of blood plasma and blood cells.

A method for washing a dispensing probe after dispensation of a specimen according to the present invention is characterized in comprising: a reading step of reading a specimen ID of a specimen to be dispensed and a specimen container ID; an extracting step of extracting analysis menu information including a specimen sample type and a specimen suction position, and specimen container information; a liquid level detecting step of detecting a specimen liquid level; a calculating step of calculating a specimen liquid surface level, a specimen suction level and a contamination adhering range of an external wall surface of a dispensing probe, based on a specimen liquid level position detected at the liquid level detecting step, the sample type, the specimen container information, and the specimen suction position; a sucking step of sucking out a specimen at the specimen suction level; a discharging step of discharging the sucked specimen to a reaction container; and a washing step of washing the dispensing probe by controlling the insertion distance and the lowering speed of the dispensing probe to the washing section and/or the amount of the washing water, based on the sample type of the specimen and the contamination adhering range of the external wall surface of the dispensing probe.

In the invention described above, the method for washing a dispensing probe according to the present invention is characterized in that the method comprises an internal wall surface washing step of performing internal wall surface washing by the discharging of an extrusion liquid from the dispensing probe, within the washing section, prior to the washing step.

A method according to the present invention is for washing a dispensing probe after dispensation of a lower layer component of a specimen which is separated into layers, the method characterized in comprising: a reading step of reading a specimen ID of a specimen to be dispensed and a specimen container ID; an extracting step of extracting analysis menu information including a specimen sample type and a specimen suction position for each layer, and specimen container information; a detecting step of detecting a specimen liquid level and an interface; a calculating step of calculating a specimen liquid surface level of each layer, a specimen suction level of each layer and a contamination adhering range of each layer of an external wall surface of a dispensing probe, based on a specimen liquid level position and an interface position detected at the detecting step, the sample type, the specimen container information, and the specimen suction position of each layer; a sucking step of sucking out a lower layer specimen at a lower layer specimen suction level; a discharging step of discharging the sucked specimen to a reaction container; a first washing step of washing the dispensing probe by controlling the insertion distance and the lowering speed of the dispensing probe to the washing section and/or the amount of the washing water, based on the sample type of the lower layer specimen and the contamination adhering range of the lower layer of the external wall surface of the dispensing probe; and a second washing step of washing the dispensing probe by controlling the insertion distance and the lowering speed of the dispensing probe to the washing section and/or the amount of the washing water, based on the sample type of the upper layer specimen and the contamination adhering range of the upper layer of the external wall surface of the dispensing probe.

Further, the method for washing a dispensing probe according to the present invention is characterized in that the method comprises an internal wall surface washing step of performing internal wall surface washing by the discharging of an extrusion liquid from the dispensing probe, within the washing section, prior to the first washing step.

### Advantageous Effects of Invention

The present invention comprises a washing control section for controlling the lowering speed and inserting distance of the dispensing section in the washing section based on the type of contamination or the like adhered to the dispensing probe, so that the present invention allows washing under optimal washing conditions, thereby cutting the washing time and reducing the amount of washing water.

### BRIEF DESCRIPTION OF THE DRAWINGS

FIG. 1 a schematic configuration diagram of an analyzer according to the Embodiments of the present invention.

FIG. 2 is a schematic configuration diagram of a specimen dispensing apparatus used in the analyzer in FIG. 1.

FIG. 3 is a schematic configuration diagram of a liquid level detecting section used in the specimen dispensing apparatus in FIG. 2.

FIG. 4 is a schematic configuration diagram of a dispensing probe washing apparatus used in the automatic analyzer in FIG. 1.

FIG. 5 is a flowchart illustrating dispensing operations for dispensing different sample types as specimens.

FIG. 6 is a flowchart illustrating dispensing operations of a normal dispensing mode in FIG. 5.

FIG. 7 is a dispensing operation diagram of a case where a blood plasma sample is dispensed.

FIG. 8 is a washing operation diagram after a blood plasma sample is dispensed.

FIG. 9 is an operation diagram of Variation Example 1 of washing after a blood plasma sample is dispensed.

FIG. 10 is an operation diagram of Variation Example 2 of washing after a blood plasma sample is dispensed.

FIG. 11 is an operation diagram of Variation Example 3 of washing after a blood plasma sample is dispensed.

FIG. 12 is a dispensing operation diagram of a case where a whole blood sample is dispensed.

FIG. 13 is a washing operation diagram after a whole blood sample is dispensed.

FIG. 14 is a flowchart illustrating dispensing operations of a special dispensing mode in FIG. 5.

FIG. 15 is a graph of pressure change when a dispensing probe is lowered into and sucks out a specimen, which is separated into layers of blood plasma and blood cells.

FIG. 16 is a diagram of a dispensing operation for dispensing blood plasma from a sample which is separated into layers of blood plasma and blood cells.

FIG. 17 is a diagram of a dispensing operation for dispensing blood cells from a sample which is separated into layers of blood plasma and blood cells.

FIG. 18 is a diagram of a washing operation after dispensation of blood cells from a sample which is separated into layers of blood plasma and blood cells.

FIG. 19 is a schematic configuration diagram of a variation example of a dispensing probe washing apparatus used in the automatic analyzer in FIG. 1.

FIG. 20 is a schematic configuration diagram of a variation example of a dispensing probe washing apparatus used in the automatic analyzer in FIG. 1.

### BEST MODE FOR CARRYING OUT THE INVENTION

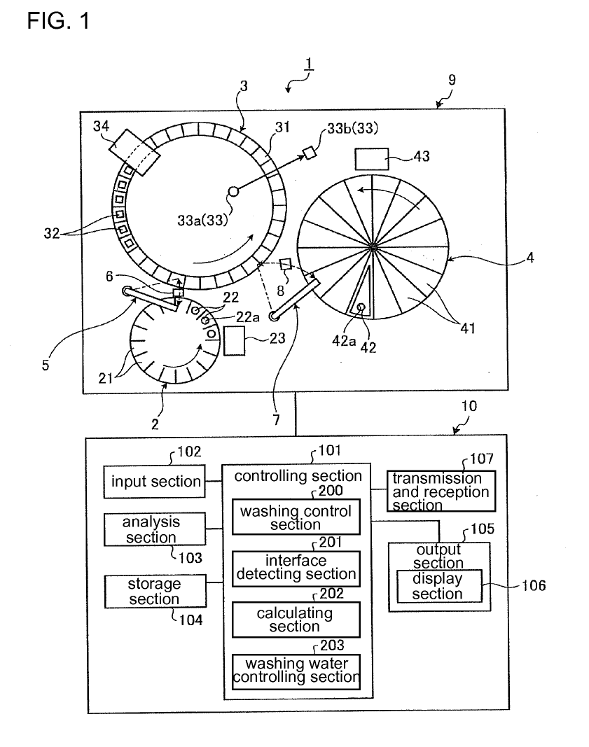

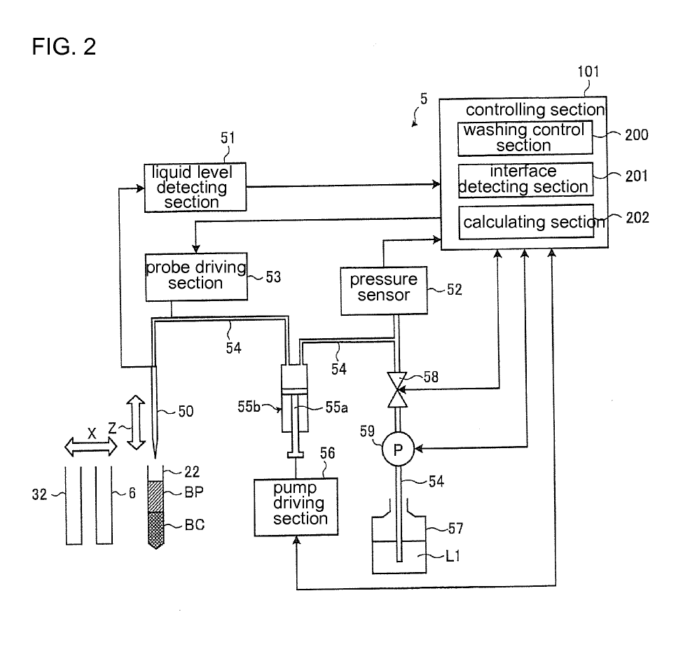

Hereinafter, preferred Embodiments of an analyzer according to the present invention will be described in detail with reference to accompanying figures. FIG. 1 is a schematic configuration diagram of an analyzer 1 according to the Embodiments. FIG. 2 is a schematic configuration diagram of a specimen dispensing apparatus 5 used in the analyzer 1 in FIG. 1.

The analyzer 1 comprises a measurement mechanism 9 for measuring light passing through a reactant in between a specimen and a reagent; and a control mechanism 10 for performing the control of the overall analyzer 1 including the measurement mechanism 9 as well as for performing analysis on measurement results of the measurement mechanism 9. The analyzer 1 automatically performs analysis on a plurality of specimens owing to the cooperation of the two mechanisms.

First, the measurement mechanism 9 will be described. The measurement mechanism 9 broadly comprises: a specimen table 2; a reaction table 3; a reagent table 4; a specimen dispensing apparatus 5; a reagent dispensing apparatus 7; and dispensing probe washing apparatuses 6 and 8.

The specimen table 2 has a disk-shaped table and comprises a plurality of specimen container housing sections 21 arranged at regular intervals along the circumferential direction of the disk-shaped table. A specimen container 22, containing a specimen, is detachably housed in each of the specimen container housing sections 21. The specimen container 22 has an opening 22a, which is upwardly open. The specimen table 2 rotates in the direction indicated by the arrow in FIG. 1, using a specimen table driving section (not shown), with a vertical line passing through the center of the specimen table 2 as an axis of rotation. As the specimen table 2 rotates, the specimen container 22 is conveyed to a specimen suction position where a specimen is sucked out by the specimen dispensing apparatus 5.

The specimen container 22 includes an identification label (now shown) adhered thereon. The identification label contains specimen information with regard to the type of the contained specimen and analysis menu therefor. In the meantime, the specimen table 2 comprises a reading section 23 for reading the information of the identification label of the specimen container 22.

The reaction table 3 has a disk-shaped table and comprises a plurality of reaction container housing sections 31 arranged at regular intervals along the circumferential direction of the disk-shaped table. A transparent reaction container 32 for containing a specimen and a reagent is detachably housed in each of the reaction container housing sections 31, in such a manner that the transparent reaction container 32 is upwardly open. The reaction table 3 rotates in the direction indicated by the arrow in FIG. 1, using a reaction table driving section (not shown), with a vertical line passing through the center of the reaction table 3 as an axis of rotation. As the reaction table 3 rotates, the reaction container 32 is conveyed to a specimen discharging position where a specimen is discharged by the specimen dispensing apparatus 5, and a reagent discharging position where a reagent is discharged by the reagent dispensing apparatus 7.

A photometry apparatus 33 has a light source 33a and a light receiving section 33b. The light source 33a outputs analysis light with a given wavelength, and the light receiving section 33b measures a beam of light, which is output from the light source 33a and is transmitted through a reaction liquid. The reaction liquid is obtained by the reaction of a specimen and a reagent and is contained in the reaction container 32. The photometry apparatus 33 is arranged such that the light source 33a and the light receiving section 33b are positioned opposite each other in a radial direction with the reaction container housing sections 31 of the reaction table 3 interposed therebetween. The reaction table 3 comprises a reaction container washing apparatus 34 for discharging a reaction liquid, which has gone through the measurement, from a reaction container 32, and for washing the reaction container 32.

The reagent table 4 has a disk-shaped table and comprises a plurality of reagent container housing sections 41 arranged at regular intervals along the circumferential direction of the disk-shaped table. A reagent container 42 containing a reagent is detachably housed in each of the reagent container housing sections 41. The reagent container 42 has an opening 42a which is upwardly open. In addition, the reagent table 4 rotates in the direction indicated by the arrow in FIG. 1, using a reagent table driving section (not shown), with a vertical line passing through the center of the reagent table 4 as an axis of rotation. As the reagent table 4 rotates, the reagent container 42 is conveyed to a reagent suction position where a reagent is sucked out by the reagent dispensing apparatus 7.

The reagent container 42 includes an identification label (now shown) adhered thereon. The identification label contains reagent information with regard to the type and the amount of the contained reagent. In the meantime, the reagent table 4 comprises a reading section 43 for reading the information of the identification label of the reagent container 42.

The specimen dispensing apparatus 5 comprises a dispensing probe for performing suction and discharge of a specimen, attached to a tip portion thereof. The specimen dispensing apparatus 5 also comprises an arm capable of freely ascending and descending in a vertical direction as well as rotating with a vertical line passing through a base section of the arm as a central axis. The specimen dispensing apparatus 5 is provided in between the specimen table 2 and the reaction table 3. The specimen dispensing apparatus 5 sucks out a specimen in a specimen container 22, which is conveyed to a given position by the specimen table 2, using the dispensing probe, turns the arm around, and dispenses the specimen into a reaction container 32, which is conveyed to a given position by the reaction table 3, to transfer the specimen into the reaction container 32 on the reaction table 3 at a given timing.

FIG. 2 is a schematic configuration diagram of a specimen dispensing apparatus 5. As illustrated in FIG. 2, the specimen dispensing apparatus 5 comprises: a dispensing probe 50; a liquid level detecting section 51; a probe driving section 53; a dispensing pump 55; a pump driving section 56; a pressure sensor 52; and a washing water pump 59.

As illustrated in FIG. 2, the dispensing probe 50 is connected with the dispensing pump 55, the pressure sensor 52 and the washing water pump 59, through tubes 54. Under the control of a washing control section 200, the dispensing probe 50 is moved in a horizontal direction as indicated with an arrow X and in a vertical direction as indicated with an arrow Z in the figure, by the probe driving section 53. Subsequently, the dispensing probe 50 sucks a specimen from a specimen container 22 successively conveyed below the dispensing probe 50, and discharges the specimen into a reaction container 32 on the reaction table 3, so that the specimen is dispensed.

The dispensing pump 55 is a syringe pump, which allows the dispensing probe 50 to suck out a specimen in a specimen container 22 and subsequently discharge the sucked specimen into a reaction container 32 conveyed by the reaction table 3. A piston 55a thereof is reciprocatively moved by the pump driving section 56.

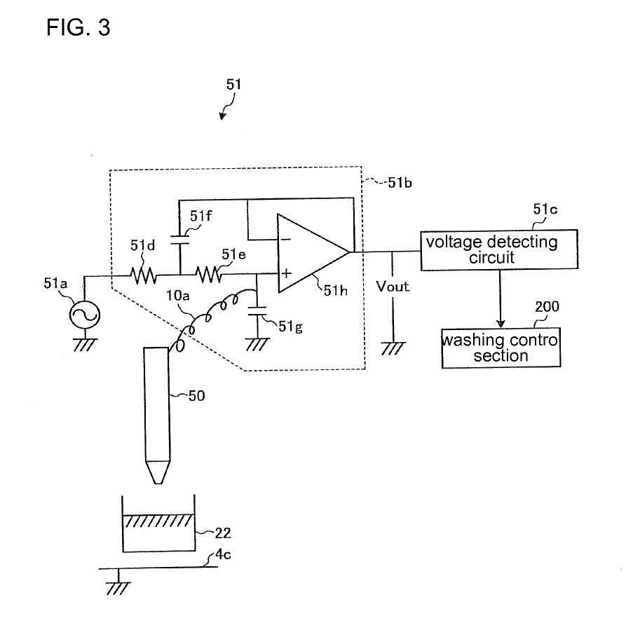

The liquid level detecting section 51 detects an interface of layers of air and a specimen (blood plasma), specifically a specimen (blood plasma) liquid level, using the change in capacitance between the dispensing probe 50 and a metal plate 4c disposed in the periphery of the specimen container 22. FIG. 3 is a schematic configuration diagram of a liquid level detecting section 51. An oscillation circuit 51a produces oscillating AC signals, and inputs the signals to a differential circuit 51b. As illustrated in FIG. 3, the differential circuit 51b has resistances 51d and 51e, capacitors 51f and 51g, and an operational amplifier 51h, and is adjusted such that the input sensitivity increases in accordance with frequencies of the oscillating AC signals produced by the oscillation circuit 51a. The positive side input terminal of the differential circuit 51b is connected to the dispensing probe 50 via a lead line 10a. A voltage detecting circuit 51c is connected to an output terminal of the differential circuit 51b, and detects output voltage Vout of the differential circuit 51b, and in accordance with the value thereof, detects whether or not a lower end of the dispensing probe 50 has touched the liquid level of a liquid in the specimen container 22. The differential circuit 51b is adjusted such that the input sensitivity increases at the frequency of the oscillation circuit 51a, in accordance with a capacitance value of a state in which the dispensing probe 50 is not in contact with the liquid level of a liquid. Thus, in the adjusted differential circuit 51b, its sensitivity decreases in accordance with a capacitance value of the state in which the dispensing probe 50 is in contact. Therefore, the voltage detecting circuit 51c detects a liquid level by the change in the output voltage Vout. The output voltage signal detected by the voltage detecting circuit 51c is output to the washing control section 200, and a specimen liquid level position (air-specimen interface) in the specimen container 22 is detected.

A calculating section 202 calculates a specimen liquid surface level and a specimen suction level, based on a liquid level position detected by the liquid level detecting section 51, specimen container information read by the reading section 23 and a set specimen suction position. The specimen suction position is set for each sample type. For example, when HbAlc is analyzed in a whole blood sample or a blood cell sample, analysis results will be influenced depending on the specimen suction position, and thus, the specimen suction position can be set by a user. In general, the specimen suction position for HbAlc is set to be an intermediate position of the specimen liquid surface level. When a specimen, such as blood plasma, is sucked out, which does not have so much influence on analysis results depending on its specimen suction position, the specimen suction position is set to be near the specimen liquid level position so that the range of an external wall surface of the dispensing probe 50 to which the specimen is adhered will be small. The specimen liquid surface level and the specimen suction level are output to the washing control section 200. The washing control section 200 then drives and controls the probe driving section 53 to allow the dispensing probe 50, which is stopped at the specimen liquid level position in the specimen container 22, to be lowered for a given distance down to the specimen suction level, and the washing control section 200 drives the pump driving section 56 to suck out and take the specimen.

The washing water pump 59 pumps up a degassed extrusion liquid L1, which is housed in a tank 57, and compresses and transports the extrusion liquid L1 to a tube 54 via an electromagnetic valve 58 provided between the washing water pump 59 and the pressure sensor 52. In such a process, by way of a control signal from a control section 101, the electromagnetic valve 58 is switched to “open” when the pumped extrusion liquid L1 is compressed and transported to the tube 54, and the dispensing probe 50 sucks out a liquid sample with the dispensing pump 55. The electromagnetic valve 58 is switched to “closed” when the liquid sample is discharged.

The pressure sensor 52 detects the pressure within the tube 54 and outputs the detected pressure as a pressure signal to an interface detecting section 201 via the control section 101.

The interface detecting section 201 detects an interface of a specimen, which is separated into layers, based on pressure changes detected by the pressure sensor 52, such as an interface of a blood plasma layer and a blood cell layer. The interface detecting section 201 is also capable of detecting an interface of air and blood plasma in a similar manner by pressure changes. A computer apparatus, for example, is used for the interface detecting section 201. The interface detecting section 201 amplifies pressure signals input from the pressure sensor 52 and performs processing for converting the pressure signals (analog) into digital signals. Subsequently, the interface detecting section 201 detects the pressure within the tube 54 from the pressure signals, which are converted into digital signals, to detect an interface position (blood cell liquid level) of a blood plasma layer and a blood cell layer. The calculating section 202 calculates a lower layer specimen (blood cell) liquid surface level and a lower layer specimen (blood cell) suction level, based on the interface position, the specimen container information read by the reading section 23, and the set specimen suction position. The calculated blood cell liquid surface level and blood cell suction level are output to the washing control section 200, and the washing control section 200 controls the driving of the probe driving section 53 to allow the dispensing probe 50, which is stopped at a blood plasma-blood cell interface in the specimen container 22, to be lowered a given distance down to a blood cell suction level. Subsequently, the pump driving section 56 is driven to suck out and draw a blood cell sample.

When a liquid level of a specimen without a layer separation, such as blood plasma, blood serum or a whole blood specimen, is detected, an air-specimen interface is detected by the liquid level detecting section 51. An air-specimen (blood plasma) interface of a specimen which is separated into layers of blood plasma, blood cells or the like is detected by the liquid level detecting section 51 or interface detecting section 201. A blood plasma-blood cell interface is detected by the interface detecting section 201. Since respective specimen suction positions are determined in accordance with the type of samples, the calculating section 202 calculates a liquid surface level and a specimen suction level based on the liquid level position or interface position detected by the liquid level detecting section 51 or the interface detecting section 201, and the specimen container information. The calculating section 202 further calculates the range for the dispensing probe 50 to be inserted into a specimen, i.e., contamination adhering range, from the calculated specimen liquid surface level and specimen suction level. In the Embodiments of the present invention, the liquid level and/or interface level are detected by means of a capacitance method (liquid level detecting section 51) and/or a pressure method (interface detecting section 201), with a high accuracy of liquid level detection. However, it is also possible to detect a liquid level using a CCD camera, or detect a liquid level and/or an interface level by measuring transmission light, which has passed through a specimen, by a light receiving section, with an LED as a light source, and based on the amount of the transmission light.

After the dispensing of a specimen into a reaction container 32 by the dispensing probe 50, the washing control section 200 controls a washing range of the dispensing probe 50 within a dispensing probe washing apparatus 6 to be described later. The washing control section 200 controls the washing range by controlling the length of the dispensing probe 50 inserted into the dispensing probe washing apparatus 6 based on the contamination adhering range on the external wall surface of the dispensing probe 50 calculated by the calculating section 202. Controlling the washing range enables the shortening of the washing time and the reduction in the amount of the washing water. The washing control section 200 also controls the lowering speed of the dispensing probe 50 to the dispensing probe washing apparatus 6 based on the sample type of the specimen read by the reading section 23. It is easier to wash blood plasma samples and blood serum samples than whole blood samples, and it is most difficult to wash blood cell samples. Therefore, the change in the lowering speed of the dispensing probe 50 into the dispensing probe washing apparatus 6 in accordance with the sample type of a specimen to be dispensed, allows the selection of washing conditions in accordance with a washing subject, thereby allowing the shortening of the washing time and the reduction in the amount of the washing water. The lowering speed of the dispensing probe 50 into the dispensing probe washing apparatus 6 is as follows: blood plasma (blood serum) samples>whole blood samples>blood cell samples. For a sample which is more difficult to wash, the lowering speed is further reduced, thereby increasing the effectiveness of the washing. The lowering speed is set such that the washing is completed when the lowering of the dispensing probe 50 into the dispensing probe washing apparatus 6 is stopped. Therefore, the washing is not a mere extension of the length of the washing time, but also the washing is performed largely while the dispensing probe 50 moves. Compared to the washing without moving of the dispensing probe 50, the exertion of the maximum washing capability is feasible for the same washing time. For a sample which is separated into layers, the lowering speed is changed to perform the washing in accordance with the type of adhered specimen contamination.

The reagent dispensing apparatus 7 comprises a dispensing probe for sucking out and discharging a reagent, attached to a tip portion thereof. The reagent dispensing apparatus 7 also comprises an arm capable of freely ascending and descending in a vertical direction as well as rotating with a vertical line passing through a base section of itself as a central axis. The reagent dispensing apparatus 7 is provided in between the reagent table 4 and the reaction table 3, and sucks out a reagent in a reagent container 42 conveyed to a given position by the reagent table 4, by the dispensing probe, turns the arm around, and dispenses the reagent into a reaction container 32, which is conveyed to a given position by the reaction table 3, to transfer the reagent into the reaction container 32 on the reaction table 3 at a given timing.

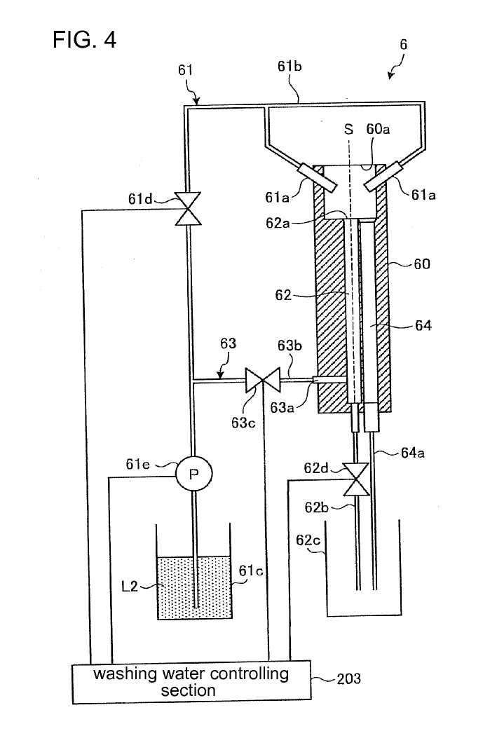

The dispensing probe washing apparatus 6 is provided in between the specimen table 2 and the reaction table 3, and at a midway position of a locus of the horizontal movement of the dispensing probe 50 in the specimen dispensing apparatus 5. In order to prevent carryover between specimens, the washing of the dispensing probe 50 is performed by the dispensing probe washing apparatus 6 every time the dispensing of a specimen is performed by the dispensing probe 50. FIG. 4 illustrates a schematic configuration diagram of a dispensing probe washing apparatus 6. The dispensing probe washing apparatus 6 has a washing vessel 60. The washing vessel 60 is formed in a tube shape, and has an opening 60a in the upper part thereof so that a lowering tip of the dispensing probe 50 is inserted therein from the above.

A reservoir vessel 62 of a prism or cylinder shape is provided at the center area of the washing vessel 60. The reservoir vessel 62 has an opening 62a at an upper part thereof so that a lowering tip of the dispensing probe 50 is inserted thereinto from the above, and a reserve washing water supplying section 63 is provided at a lower portion of the side surface thereof. The reserve washing water supplying section 63 is connected with the reservoir vessel 62 via a nozzle portion 63a, and the nozzle portion 63a is provided with its outlet facing the inward of the reservoir vessel 62. The nozzle portion 63a is connected with one end of a tube 63b, and the other end of the tube 63b is connected to a tank 61c via an electromagnetic valve 63c and a pump 61e. The tank 61c is for housing washing water L2. In addition, spout washing water supplying section 61 is provided at an upper part of the washing vessel 60. The spout washing water supplying section 61 has a nozzle section 61a. The nozzle section 61a is provided at the upper part inside the washing vessel 60 and directs its outlets diagonally below, and a plurality of the nozzle sections 61a (two outlets in the present Embodiment) are provided towards a perpendicular center line S of the washing vessel 60. Each of the nozzle sections 61a is connected with one end diverged from a tube 61b. The tube 61b is formed such that the one and the other end join as one in the midway of the tube. The other end of the tube 61b is connected to the tank 61c for housing washing water L2, via an electromagnetic valve 61d and a pump 61e. The tube 61b, which connects the tank 61c for housing the washing water L2 with the pump 61e, diverges into the tube 63b for supplying the washing water L2 to the reserve washing water supplying section 63, and into the spout washing water supplying section 61. Distilled water, degassed water or the like is used as the washing water L2. In addition, one end of a tube 62b is connected to a bottom part of the reservoir vessel 62, and the other end of the tube 62b is connected to a disposal tank 62c via an electromagnetic valve 62d.

The washing vessel 60 comprises an overflow vessel 64 provided therein. The overflow vessel 64 is disposed in parallel with the reservoir vessel 62 inside the washing vessel 60. The opening of the overflow vessel 64 is formed in a conical shape such that the opening forms an inclined surface downwards from the opening 62a of the reservoir vessel 62, and the opening of the overflow vessel 64 is further formed such that the bottom part thereof penetrates the bottom part of the washing vessel 60. One end of a tube 64a is connected to a lower part of the overflow vessel 64. The other end of the tube 64a is connected to the disposal tank 62c.

The dispensing probe washing apparatus 6 opens the electromagnetic valve 61d and drives the pump 61e, so that the washing water L2 housed in the tank 61c is spouted into the washing vessel 60 from the outlet of the nozzle section 61a via the tube 61b. In addition, the dispensing probe washing apparatus 6 opens the electromagnetic valve 63c and drives the pump 61e, so that the washing water L2 housed in the tank 61c is supplied into the reservoir vessel 62 from the outlet of the nozzle section 63a via the tube 63b, and the washing water L2 is also reserved in the reservoir vessel 62. The washing water L2 spouted from the nozzle section 61a into the inside the washing vessel 60 as well as the washing water L2 supplied from the nozzle section 63a into the reservoir vessel 62 and overflow from the opening 62a of the reservoir vessel 62, are allowed to overflow into the overflow vessel 64. Since the wall surface between the reservoir vessel 62 and the overflow vessel 64 is in such a shape that forms an inclined surface inclining downwards from the reservoir vessel 62 to the overflow vessel 64, the washing water L2 is guided from the opening 62a along the inclined surface into the overflow vessel 64. The overflow washing water L2 or the like is discharged from the overflow vessel 64 via the tube 64a to the disposal tank 62c, which is located outside the washing vessel 60. In addition, as the electromagnetic valve 62d is opened, the washing water L2 reserved in the reservoir vessel 62 is discharged via the tube 62b to the disposal tank 62c.

A washing water controlling section 203 controls an overflow timing of the washing water L2 reserved in the reservoir vessel 62 to the overflow vessel 64. The washing water controlling section 203 also controls the supplying amount of the washing water L2 supplied from the reserve washing water supplying section 63 based on the sample type of the specimen to be washed. The washing water controlling section 203 controls the supply amount of the washing water L2 to be larger as the sample type becomes a more difficult one to be washed. Therefore, the washing water controlling section 203 controls the amount of the washing water to be larger in the order of the blood plasma (blood serum) samples<whole blood samples<blood cell samples. However, if some extended washing time can be taken, sufficient washing is feasible only by adjusting the lowering speed of the dispensing probe 50 to the washing vessel 60 by the washing control section 200, without the controlling of adjustment of the washing water amount by the washing water controlling section 203 in accordance with the sample type of specimens to be washed.

The dispensing probe washing apparatus 8 is provided in between the reaction table 4 and the reaction table 3, and at a midway position of a locus of the horizontal movement of the dispensing probe in the reagent dispensing apparatus 7. In order to prevent carryover between specimens, the washing of the dispensing probe is performed by the dispensing probe washing apparatus 8 every time the dispensing of a specimen is performed by the dispensing probe.

Next, the control mechanism 10 will be described. As illustrated in FIG. 1, the control mechanism 10 comprises a control section 101, an input section 102, an analysis section 103, a storage section 104, an output section 105 and a transmission and reception section 107. The respective sections comprised by the control mechanism 10 are electrically connected to the control section 101. The control section 101 is configured with a CPU and the like, for controlling the processing and operation of the respective sections of the automatic analyzer 1. The control section 101 performs given input and output controls of information that is input to and output from the respective configuration parts, and performs given information processing on the information. The control section 101 also comprises: a washing control section 200 for controlling the specimen dispensing apparatus 5; a interface detecting section 201 for detecting an interface of a specimen which is separated into layers; a calculating section 202 for calculating a contamination adhering range on an external wall surface of the dispensing probe 50; and a washing water controlling section 203 for controlling the dispensing probe washing apparatus 6. The analysis section 103 is connected to the photometry apparatus 33 via the control section 101, for analyzing a concentration of components of a specimen or the like based on the amount of light received by the light receiving section 33b, and outputting the analysis result to the control section 101. The input section 102 is a part for performing operations of inputting an examination menu or the like to the control section 101. For example, a keyboard, a mouse or the like is used as the input section 102. The washing control section 200, interface detecting section 201 and calculating section 202 may be comprised by the specimen dispensing apparatus 5; and the washing water controlling section 203 may be comprised by the dispensing probe washing apparatus 6.

The storage section 104 is configured with a hard disk for magnetically storing information, and a memory for loading and electrically storing various programs for processing from the hard disk when the automatic analyzer 1 performs the processing. The storage section 104 stores various types of information including analysis results or the like of a specimen. The storage section 104 may also comprise an auxiliary storage unit capable of reading information stored in a storage medium, such as a CD-ROM, a DVD-ROM and a PC card. The storage section 104 also stores information required for the analysis of all the analyzable analysis menu, such as analysis conditions including a sample type of specimens, an analysis menu, and a specimen dispensing amount and the like, as well as specimen container information including a specimen container shape corresponding to a specimen container type.

The output section 105 is configured with a printer, a speaker or the like, and outputs various types of information associated with analysis, under the control of the control section 101. The output section 105 comprises a display section 106 configured with a display or the like. The display section 106 is for displaying analysis content, a warning or the like, and a display panel or the like is used for the display section 106. The input section 102 and the display section 106 may also be achieved with a touch panel. The transmission and reception section 107 has a function as an interface for transmitting and receiving information in accordance with a given format via a communication network (not shown). Analysis information such as the sample type of specimens, an analysis menu and the dispensing amount of specimens may be collectively managed by a host computer (not shown) instead of the storage section 104 described above. In such a case, the transmission and reception section 107 transmits and receives such information to extract analysis information.

In the automatic analyzer 1 with such a configuration, the specimen dispensing apparatus 5 dispenses a specimen from a specimen container 22 to a reaction container 32. In addition, the reagent dispensing apparatus 7 dispenses a reagent from a reagent container 42 to the reaction container 32. While the reaction container 32, into which the specimen and reagent have been dispensed, is conveyed by the reaction table 3 along the circumferential direction, the specimen and the reagent are stirred and they react with each other, passing through the light source 33a and the light receiving section 33b. At this stage, analysis light is output from the light source 33a and the analysis light passes through a reaction liquid inside the reaction container 32, and the analysis light is measured by the light receiving section 33b to analyze component concentration or the like. The reaction liquid after the measurement is discharged by the reaction container washing apparatus 34 from the reaction container 32 finished with the analysis, and the reaction container 32 is washed by the reaction container washing apparatus 34. Subsequently, the reaction container 32 is used again for analysis of a specimen. The dispensing probe 50 after the dispensing of a specimen is washed by the dispensing probe washing apparatus 6 every time dispensing is performed in order to prevent the carryover between specimens or to prevent clogging or contamination adhesion of the dispensing probe due to specimen components.

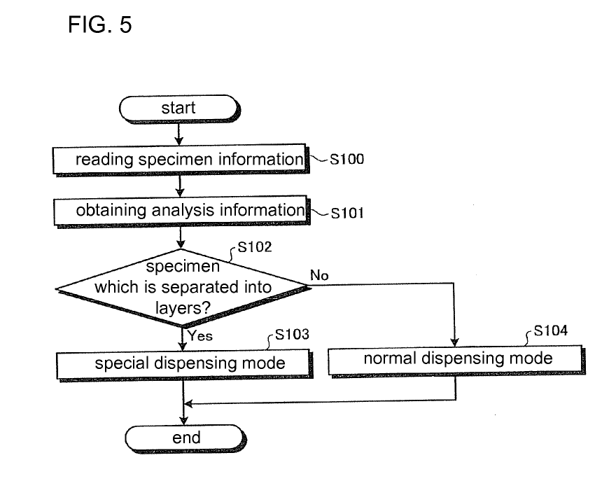

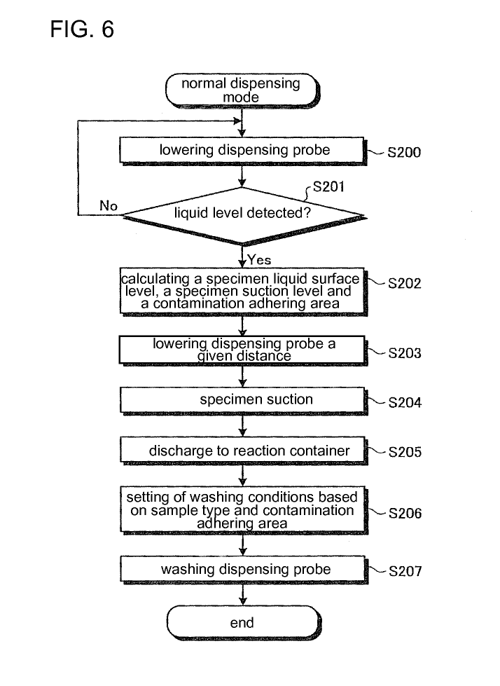

Next, dispensing operations in dispensing different sample types as specimens by a dispensing probe 50 will be described. FIG. 5 is a flowchart illustrating dispensing operations for dispensing different sample types as specimens. FIG. 6 is a flowchart illustrating dispensing operations of a normal dispensing mode in FIG. 5.

As illustrated in FIG. 5, the reading section 23 first reads a specimen ID of a specimen to be dispensed, and a specimen container ID from an identification label of a specimen container 22 (Step S100). Analysis information such as the type of the specimen, a specimen suction position, and an analysis menu, as well as specimen container information including the shape of a specimen container, is obtained from the storage section 104 based on the read specimen ID and specimen container ID (Step S101). The control section 101 switches dispensing modes based on information on the type of the specimen, i.e., information as to whether the specimen is separated into layers (Step S102). If the specimen is the one which is separated into layers of blood plasma and blood cells (Step S102: Yes), then the dispensing is performed in a special dispensing mode (Step S103). If the specimen does not have layer separation, such as other blood plasmas or whole blood specimens (Step S102: No), then a control section 15 performs a normal dispensing mode and ends the present controlling (Step S104).

As illustrated in FIG. 6, in the normal dispensing mode (Step S104), under the control by the washing control section 200, the dispensing probe 50 is lowered into the specimen container 22 (Step S200). The dispensing probe is lowered until the liquid level detecting section 51 detects a liquid level (Step S201: No). After the detection of a liquid level (Step S201: Yes), the lowering of the dispensing probe 50 is stopped. Subsequently, the calculating section 202 calculates a specimen liquid surface level, a specimen suction level and a contamination adhering range, based on the sample type, specimen suction position, specimen container information and liquid level position (Step S202). Since the specimen suction position and the specimen liquid surface level are different depending on the sample type and the specimen, the specimen liquid surface level, specimen suction level and contamination adhering range are calculated for each specimen. The specimen liquid surface level is a specimen liquid surface level in a specimen container 22, which is calculated from the specimen container information and liquid level position. The specimen suction level is a level at which a specimen is sucked out within the specimen, which is calculated from a specimen suction position and a specimen liquid surface level set for each sample. If it is set to perform the suction from the lower most part depending on the sample type, then the specimen suction level is calculated from the specimen container information. The contamination adhering range is a specimen adhering range to the external wall of the dispensing probe 50, which is calculated from the specimen liquid surface level and the specimen suction level.

The dispensing probe 50 is lowered a given distance from the liquid level position to the specimen suction level (Step S203). Subsequently, the specimen is sucked out (Step S204), and the sucked specimen is discharged into the reaction container 32 (Step S205). Subsequently, based on sample type information of the specimen obtained at Step S101 and the contamination adhering area calculated at Step S202, the washing control section 200 and the washing water controlling section 203 set washing conditions, such as a washing range, a lowering speed and/or washing water amount, of the dispensing probe 50 in the dispensing probe washing apparatus 6 (Step S206). After the dispensing probe 50 is conveyed to the dispensing probe washing apparatus 6, the dispensing probe 50 is washed under the washing conditions set at Step S206 (Step S207). The washing water controlling section 203 controls the supply amount of the washing water L2 from the spout washing water supplying section 61 and the reserve washing water supplying section 63 in the dispensing probe washing apparatus 6, as well as the overflow amount and overflow timing from the reservoir vessel 62 to the overflow vessel 64. For each sample type of the specimens, the washing control section 200 and the washing water controlling section 203 control the washing conditions to perform such washing.

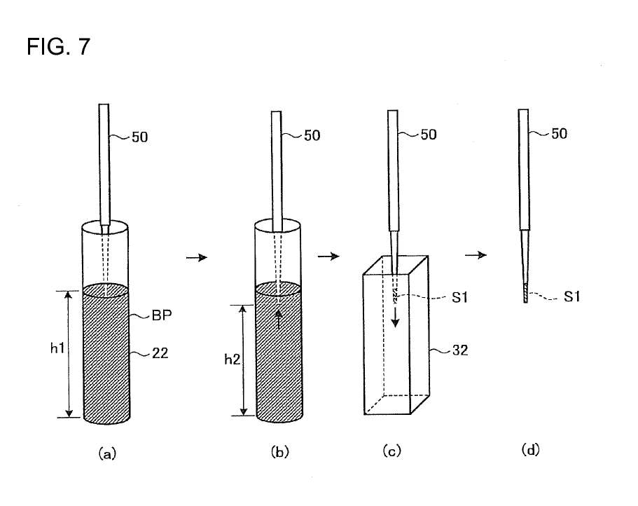

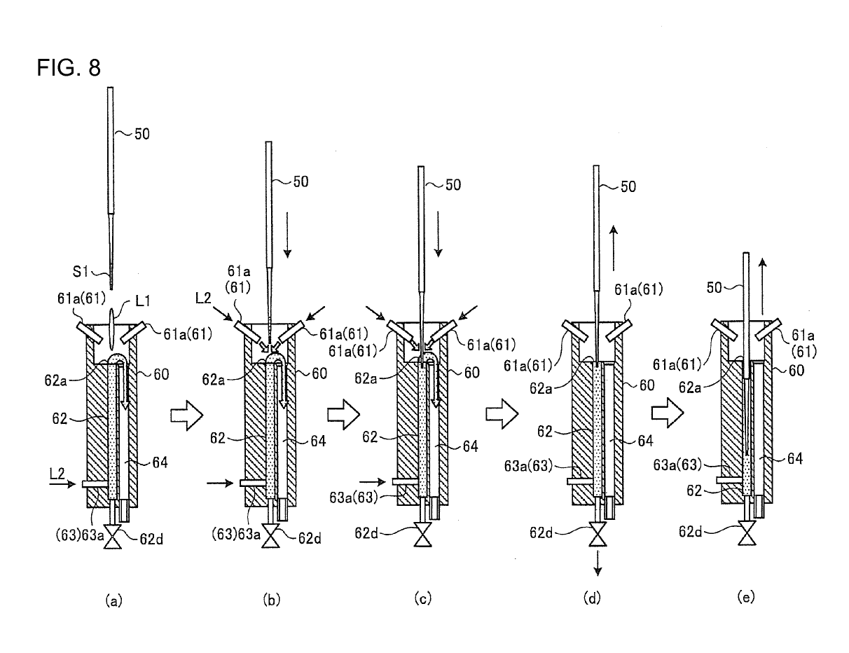

Next, the dispensing and washing of specimens for each sample type will be described for individual cases. FIG. 7 is a dispensing operation diagram of a case where a blood plasma sample is dispensed. FIG. 8 is a washing operation diagram after a blood plasma sample BP is dispensed. As illustrated in FIG. 7(a), when the dispensing probe 50 is lowered into a specimen container 22 and the liquid level detecting section 51 detects a liquid level, the lowering of the dispensing probe 50 is paused. Also, based on a detected blood plasma liquid level position, a set blood plasma suction position, and specimen container information, the calculating section 202 calculates a blood plasma liquid surface level h₁ and a blood plasma suction level h₂. As illustrated in FIG. 7(b), under the control of the washing control section 200, the dispensing probe 50 is lowered a given distance from the blood plasma liquid surface level to the blood plasma suction level h₂ and the blood plasma BP is sucked out. The sucked blood plasma BP is discharged into a reaction container 32 (see FIG. 7(c)), and the blood plasma BP is adhered to the external wall surface of the dispensing probe 50 by a range S₁, which corresponds to the length of the dispensing probe 50 inserted in the blood plasma BP. The calculating section 202 calculates the area of the contamination adhering range S₁ based on the blood plasma liquid surface level h₁ and the blood plasma suction level h₂ (see FIG. 7(d)).

The blood plasma specimen BP is dispensed in the reaction container 32, and subsequently, the dispensing probe 50 is conveyed to the dispensing probe washing apparatus 6 and the washing of an internal surface of the dispensing probe 50 is first performed, as illustrated in FIG. 8(a). The pump driving section 56 is driven to allow the piston 55a to move and advance, so that the extrusion liquid L1 is discharged from the dispensing probe 50 together with the blood plasma BP remaining inside the dispensing probe 50. At this stage, the reservoir vessel 62 is controlled by the washing water controlling section 203 such that at least the washing water L2 is supplied to the reservoir vessel 62 from the reserve washing water supplying section 63 to be allowed to overflow along the inclined surface formed between the reservoir vessel 62 and the overflow vessel 64, from the opening 62a of the reservoir vessel 62, into the overflow vessel 64, before the extrusion liquid L1 falls into and reach the reservoir vessel 62 by the advancement of the piston 55a. During the falling of the extrusion liquid L1 into the reservoir vessel 62, it is controlled such that the overflow goes from the reservoir vessel 62 into the overflow vessel 64. By this overflowing, the discharged extrusion liquid L1 including the blood plasma specimen BP is discharged to the overflow vessel 64 together with the overflow washing water L2, and the degree of cleanliness of the washing water L2 within the reservoir vessel 62 is maintained.

As the discharging of the extrusion liquid L1 is stopped, the washing of the internal wall surface of the dispensing probe 50 ends, and the washing of the external wall surface of the dispensing probe 50 is performed. In the external wall surface of the dispensing probe 50 after the dispensing of the blood plasma specimen, the blood plasma BP is adhered by the contamination adhering range S₁ due to the lowering into the blood plasma BP. Since blood plasma is a sample type of specimen that is easy to wash off, the washing control section 200 sets the lowering speed of the dispensing probe 50 to the washing vessel 60 to be high, and sets the washing range to be the contamination adhering range S₁ plus some extra. The dispensing probe 50 is allowed to be lowered and advanced, at a high speed, in the flow path created by spouting the washing water L2 from the nozzle section 61a of the spout washing water supplying section 61, at an upper part of the reservoir vessel 62 from which the washing water L2 is allowed to overflow, and the external wall surface of the dispensing probe 50 is washed (see FIG. 8(b)). In a state where the washing water L2 is supplied from the nozzle section 63a of the reserve washing water supplying section 63 and the washing water L2 is allowed to overflow from the opening 62a of the reservoir vessel 62 into the overflow vessel 64, the dispensing probe 50 is allowed to be lowered and advanced, at a high speed, to be inserted into the opening of the washing vessel 60. The washing water L2 injected out of the nozzle section 61a hits the external wall surface of the dispensing probe 50 along the longitudinal direction (advancing direction) of the dispensing probe 50, and the specimen adhered to the external wall surface of the dispensing probe 50 is removed, thus washing the external wall surface of the dispensing probe 50. Although the removed specimen falls into the reservoir vessel 62 together with the washing water L2, the reservoir vessel 62 overflows the washing water L2 from the opening 62a. Thus, the washing water L2 injected from the nozzle section 61a is discharged to the overflow vessel 64 together with the overflow washing water L2 and the degree of cleanliness of the washing water L2 within the reservoir vessel 62 is maintained.

When the dispensing probe 50 is lowered at a high speed, the washing water controlling section 203 performs control such that the washing water L2 is spouted from the nozzle section 61a of the spout washing water supplying section 61, from the time at which the tip section of the dispensing probe is expected to advance into the washing water L2 being spouted until the time at which an upper part of the external wall surface above the contamination adhering range S₁ of the dispensing probe is expected to advance into the washing water L2 being spouted. Further, during the time which the washing water L2 from the nozzle section 61a drops into the reservoir vessel 62, the washing water controlling section 203 performs control such that the washing water L2 is supplied from the reserve washing water supplying section 63 and the washing water L2 is allowed to overflow to the overflow vessel 64.

After the washing with the spouting of the washing water L2 from the nozzle section 61a, the contamination adhering range S₁ of the dispensing probe 50 is dipped in the washing water L2 reserved in the reservoir vessel 62 to further wash the external wall surface (see FIG. 8(c)). The washing water controlling section 203 performs control such that the washing water L2 is supplied from the reserve washing water supplying section 63 and the washing water L2 overflows to the overflow vessel 64 even while the contamination adhering range S₁ of the dispensing probe 50 is lowered and dipped in the washing water L2 reserved in the reservoir vessel 62. As a result, the degree of the cleanliness of the washing water L2 within the reservoir vessel 62 is maintained, and the washing capability of the dispensing probe 50 by the dip-washing is improved.

After the dispensing probe 50 is lowered and dipped in the washing water L2 reserved in the reservoir vessel 62, the washing water controlling section 203 stops the supply of the washing water L2 from the reserve washing water supplying section 63 to the reservoir vessel 62, and allows the dispensing probe 50 to ascend and to be pulled out of the washing water L2 reserved in the reservoir vessel 62 (see FIG. 8(d)). During the pulling up of the dispensing probe 50, the overflow from the reservoir vessel 62 is stopped, and the pulling speed is controlled, so that the amount of the washing water L2 adhered to the dispensing probe 50 can be reduced. After the pulling up of the dispensing probe 50 from the washing water L2 in the reservoir vessel 62, the electromagnetic valve 62d is operated to be opened, so that the washing water L2 in the reservoir vessel 62 is discharged into the disposal tank 62c, and the washing ends (see FIG. 8(e)).

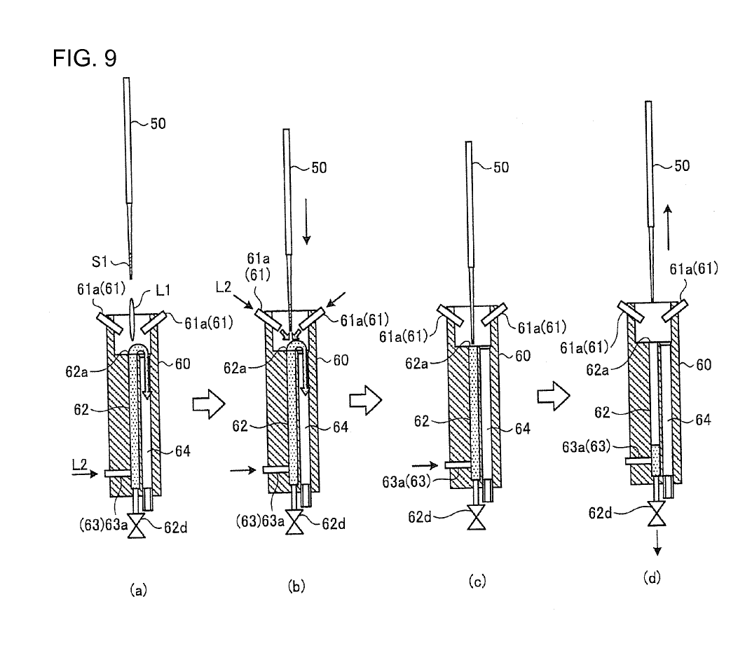

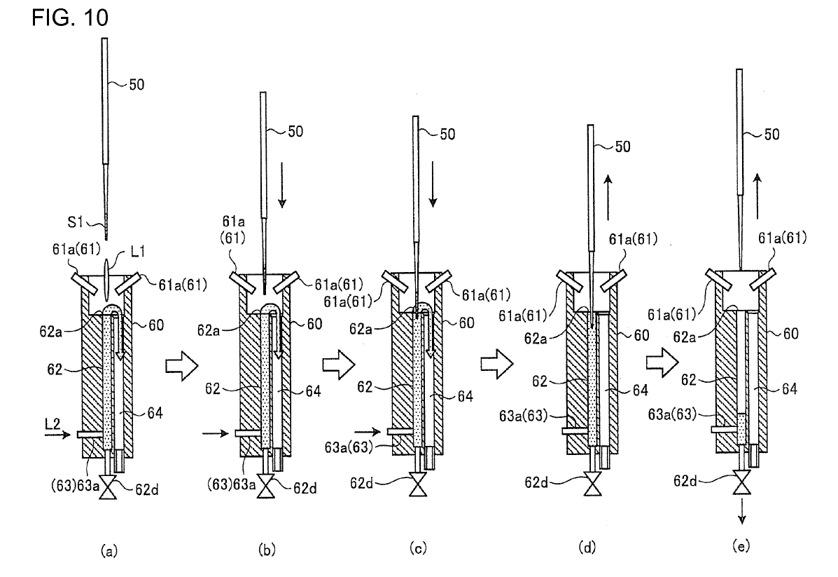

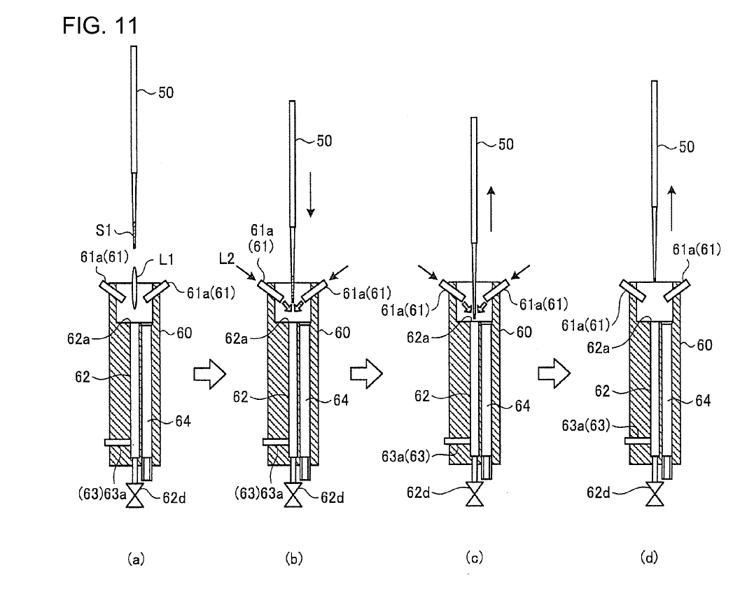

When the type of sample that is easy to wash, such as blood plasma BP (blood serum BS), is washed off, the washing may be performed as illustrated in FIGS. 9 to 11, instead of the washing method described above. By using both the spout-washing of the washing water L2 by the spout washing water supplying section 61 and the dip-washing like the above-mentioned method, the washing effect can be increased and carryover can be prevented. However, a high level analysis accuracy may not be required depending on specimen to be analyzed or an analysis menu. In such a case, the washing time and the amount of washing water to be used can be reduced by applying the washing method illustrated in FIGS. 9 to 11. FIG. 9 illustrates a washing method that is different from the above-mentioned method in that the dispensing probe 50 is not dipped in the washing water L2 reserved in the reservoir vessel 62 to be washed (see FIG. 9(c)). FIG. 10 illustrates a washing method in which the dispensing probe 50 is dipped in the washing water L2 reserved in the reservoir vessel 62 and is washed without using the spouting of the washing water L2 from the nozzle section 61a of the spout washing water supplying section 61 (see FIG. 10(d)). FIG. 11 illustrates a method for washing the dispensing probe 50 only with the spouting of the washing water L2 from the nozzle section 61a of the spout washing water supplying section 61, without reserving the washing water L2 in the reservoir vessel 62. In this method, it is not necessary to perform the internal wall washing by the spouting of the extrusion liquid L1 prior to the external wall washing, which may be performed together with the washing by the spouting of the washing water L2 from the nozzle section 61a, or after the washing by the spouting of the washing water L2 from the nozzle section 61a (see FIG. 11(c)). In any of the washing methods illustrated in FIGS. 9 to 11, the washing control section 200 controls the lowering speed of the dispensing probe 50 down to the washing vessel 60 to be greater, thereby shortening the washing time and reducing the amount of the washing water to be used. In addition, when the type of sample that is easy to wash, such as blood plasma BP (blood serum), is washed off, the washing water controlling section 203 may control the supply amount of the washing water L2 to be smaller from the nozzle section 61a of the spout washing water supplying section 61 and/or the nozzle section 63a of the reserve washing water supplying section 63.

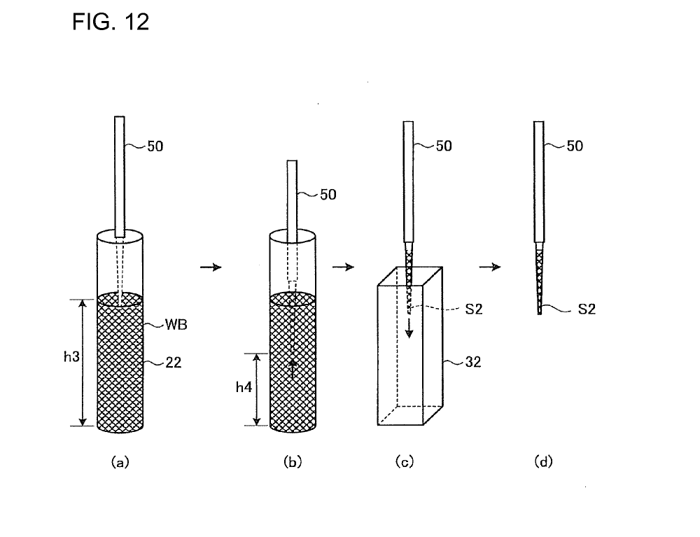

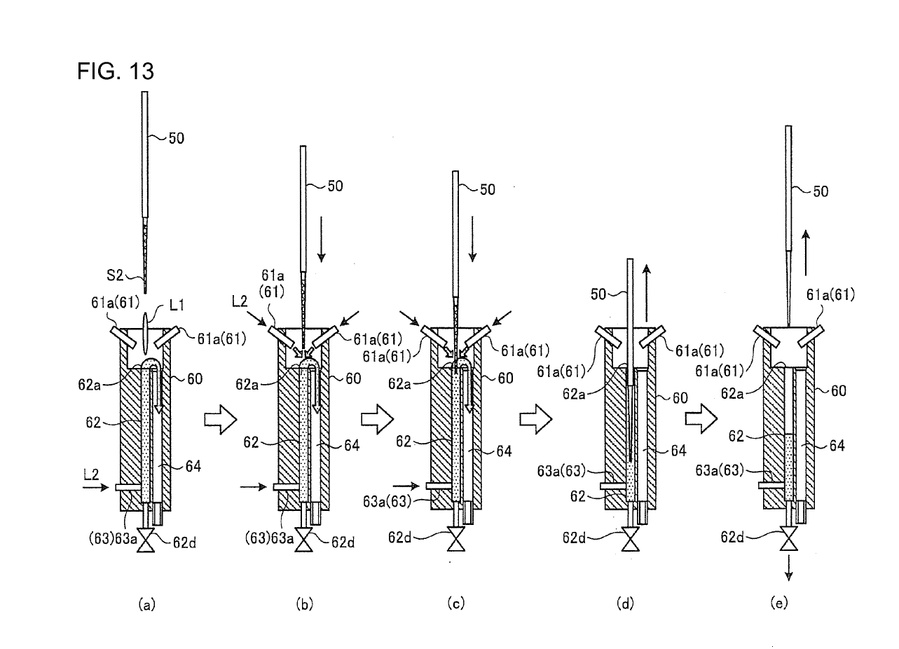

Next, the dispensing and washing in the case where the specimen is a whole blood specimen WB will be individually described. FIG. 12 is a diagram of a dispensing operation for dispensing a whole blood specimen WB. FIG. 13 is a diagram of a washing operation after a whole blood specimen WB is dispensed. As illustrated in FIG. 12(a), when the dispensing probe 50 is lowered into the specimen container 22 and the liquid level detecting section 51 detects a liquid level, the lowering of the dispensing probe 50 is paused. Based on the detected whole blood liquid level position, a set whole blood specimen suction position, and specimen container information, the calculating section 202 calculates a whole blood liquid surface level h₃ and a whole blood suction level h₄. As illustrated in FIG. 12(b), under the control by the washing control section 200, the dispensing probe 50 is lowered a given distance from the whole blood liquid level position to the whole blood suction level h₄, and the whole blood specimen WB is sucked out. The sucked out whole blood specimen WB is discharged to a reaction container 32 (see FIG. 12(c)). The whole blood WB corresponding to the length of the dispensing probe 50 inserted into the whole blood specimen is adhered to the external wall surface of the dispensing probe 50 after the discharge of the whole blood, and the calculating section 202 calculates an area of a contamination adhering range S₂ based on the whole blood liquid surface level h₃ and the whole blood suction level h₄ (see FIG. 12(d)).

After the whole blood specimen WB is dispensed in the reaction container 32, the dispensing probe 50 is conveyed to the dispensing probe washing apparatus 6 as illustrated in FIG. 13(a), and washing for the internal wall surface of the dispensing probe 50 is first performed. By the driving of the pump driving section 56 and the moving of the piston 55a is advancing, the extrusion liquid L1 is made to be discharged from the dispensing probe 50 together with the whole blood WB remaining inside the dispensing probe 50. At this stage, the reservoir vessel 62 is controlled by the washing water controlling section 203 such that at least the washing water L2 is supplied from the reserve washing water supplying section 63 before the extrusion liquid L1 falls into and reaches the reservoir vessel 62 by the advancement of the piston 55a, to be allowed to overflow along the inclined surface formed between the reservoir vessel 62 and the overflow vessel 64, from the opening 62a of the reservoir vessel 62 to the overflow vessel 64. When the extrusion liquid L1 is falling into the reservoir vessel 62, it is controlled to overflow from the reservoir vessel 62 to the overflow vessel 64. By this overflow, the discharged extrusion liquid L1 including the whole blood specimen is discharged to the overflow vessel 64 together with the overflow washing water L2, thus maintaining the degree of cleanliness of the washing water L2 in the reservoir vessel 62.

With the stop of the discharging of the extrusion liquid L1, the washing of the internal wall surface of the dispensing probe 50 ends, and the washing of the external wall surface of the dispensing probe 50 is then performed. The whole blood WB of the area equal to the contamination adhering range S₂ is adhered to the external wall surface of the dispensing probe 50 after the dispensing of the whole blood specimen, due to the lowering into the whole blood specimen WB. Since the whole blood specimen WB is more difficult to wash compared to the blood plasma BP (blood serum), the washing control section 200 sets the lowering speed of the dispensing probe 50 into the washing vessel 60 to be a medium speed, and sets the washing range to be a contamination adhering range S₂ plus some extra. The dispensing probe 50 is allowed to be lowered and advanced, at a medium speed, in the flow path created by spouting the washing water L2 from the nozzle section 61a of the spout washing water supplying section 61, at an upper part of the reservoir vessel 62 from which the washing water L2 is allowed to overflow, and the whole blood WB adhered to the external wall surface of the dispensing probe 50 is washed (see FIG. 13(b)). In a state where the washing water L2 is supplied from the nozzle section 63a of the reserve washing water supplying section 63 and the washing water L2 is allowed to overflow from the opening 62a of the reservoir vessel 62 into the overflow vessel 64, the dispensing probe 50 is allowed to be lowered and advanced, at a medium speed, to be inserted into the opening 60a of the washing vessel 60. The washing water L2 injected out of the nozzle section 61a hits the external wall surface of the dispensing probe 50 along the longitudinal direction (advancing direction) of the dispensing probe 50, and the whole blood specimen WB adhered to the external wall surface of the dispensing probe 50 is removed, thus washing the external wall surface of the dispensing probe 50. Although the removed specimen falls into the reservoir vessel 62 together with the washing water L2, the reservoir vessel 62 is overflows the washing water L2 from the opening 62a. Thus, the washing water L2 injected from the nozzle section 61a is spouted to the overflow vessel 64 together with the overflow washing water L2 and the degree of cleanliness of the washing water L2 within the reservoir vessel 62 is maintained.

When the dispensing probe 50 is lowered at a speed of a medium degree, the washing water controlling section 203 performs control such that the washing water L2 is spouted from the nozzle section 61a of the spout washing water supplying section 61, from the time at which the tip section of the dispensing probe is expected to advance into the washing water L2 being spouted until the time at which an upper part of the external wall surface above the contamination adhering range S₂ of the dispensing probe 50 is expected to advance into the washing water L2 being spouted and a certain period of time elapses. Further, during the time which the washing water L2 from the nozzle section 61a drops into the reservoir vessel 62, the washing water controlling section 203 performs control such that the washing water L2 is supplied from the reserve washing water supplying section 63 and the washing water L2 overflows to the overflow vessel 64. As a result, the degree of the cleanliness of the washing water L2 within the reservoir vessel 62 is maintained, and the washing capability of the dispensing probe 50 by the dip-washing is improved.

During the washing by the spouting of the washing water L2 from the nozzle section 61a of the spout washing water supplying section 61, under the control of the washing control section 200, the dispensing probe 50 is lowered at a medium speed and the dispensing probe 50 is inserted and dipped in the reservoir vessel 62, from which the washing water L2 is allowed to overflow to the overflow vessel 64 by the supply of the washing water L2 from the reserve washing water supplying section 63. Even after the washing with the spouting of the washing water L2 from the nozzle section 61a of the spout washing water supplying section 61 ends, the washing control section 200 allows the dispensing probe 50 to be lowered at a medium speed until the contamination adhering range S₂ of the external wall surface of the dispensing probe 50 is completely inserted and dipped in the washing water L2 in the reservoir vessel 62 (contamination adhering range S₂ plus some extra). The washing water controlling section 203 performs control such that the washing water L2 is supplied from the reserve washing water supplying section 63 and the washing water L2 overflows to the overflow vessel 64 even while the contamination adhering range S₂ of the dispensing probe 50 is being lowered and dipped in the washing water L2 reserved in the reservoir vessel 62.

After the contamination adhering range S₂ of the dispensing probe 50 is completely dipped in the washing water L2 reserved in the reservoir vessel 62, the washing water controlling section 203 stops the supply of the washing water L2 from the reserve washing water supplying section 63 to the reservoir vessel 62, and the washing control section 200 allows the dispensing probe 50 to ascend and to be pulled out of the washing water L2 reserved in the reservoir vessel 62 (see FIG. 13(d)). During the pulling up of the dispensing probe 50, the overflow from the reservoir vessel 62 is stopped, and the pulling speed is controlled, so that the amount of the washing water L2 adhered to the dispensing probe 50 can be reduced. After the pulling up of the dispensing probe 50 from the washing water L2 in the reservoir vessel 62, the electromagnetic valve 62d is operated to be opened, so that the washing water L2 in the reservoir vessel 62 is discharged into the disposal tank 62c, and the washing ends (see FIG. 13(e)).

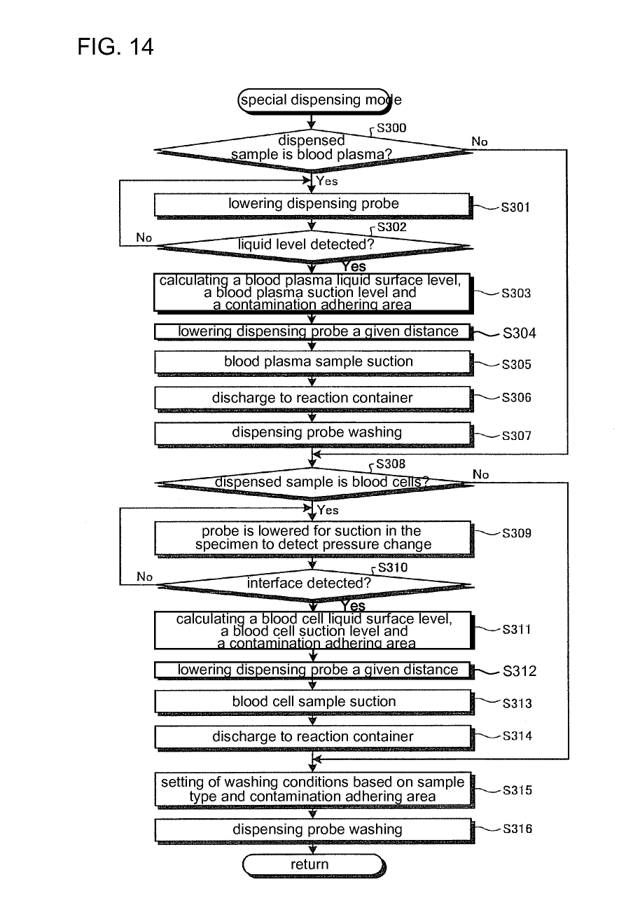

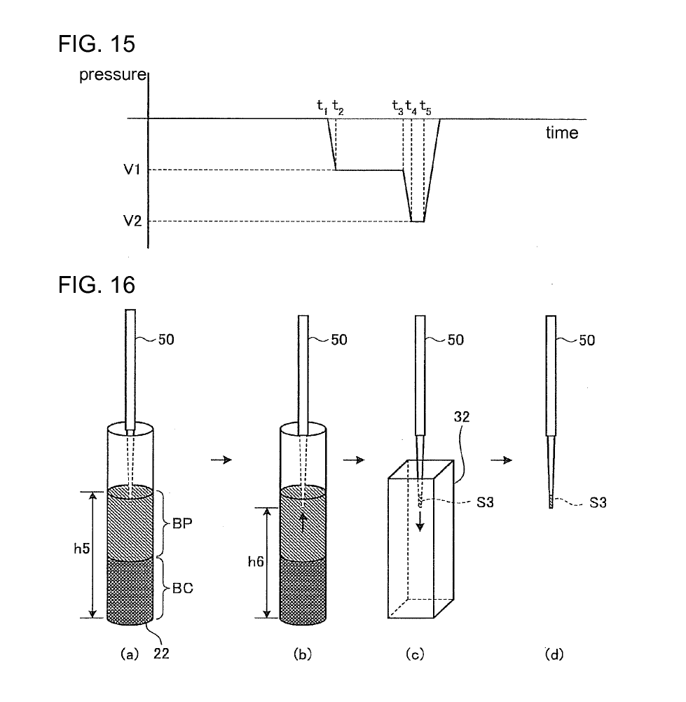

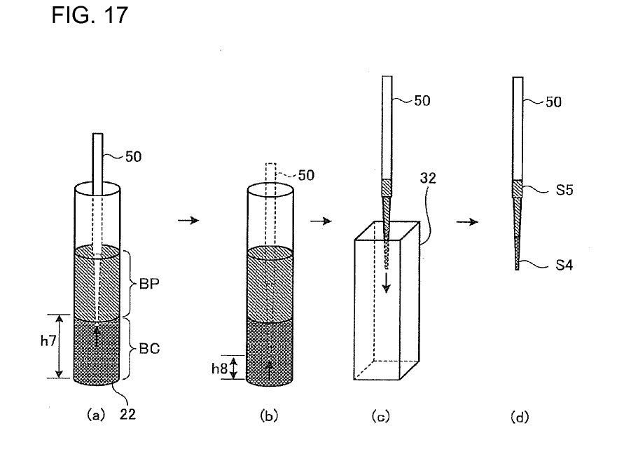

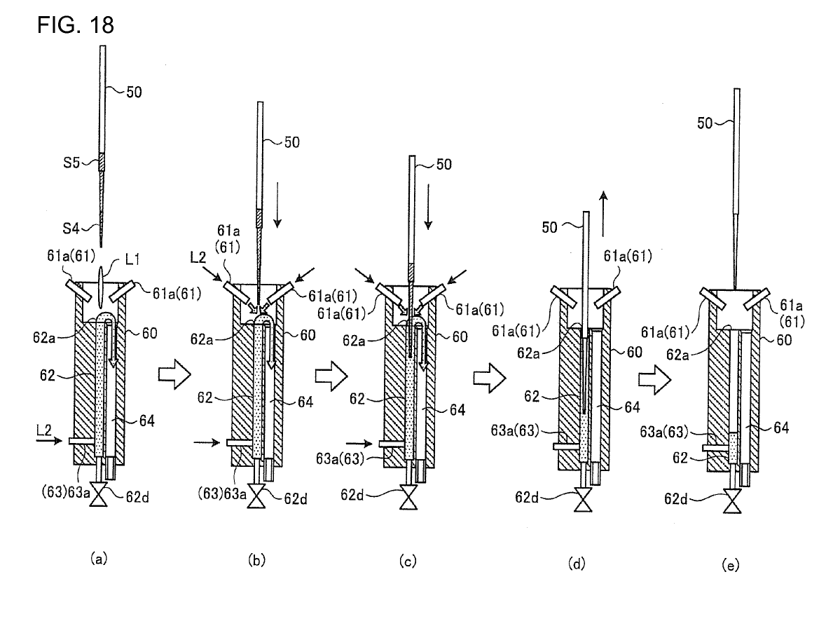

Next, in a special dispensing mode, the specimen which is the one separated into layers of blood plasma and blood cells, will be described. FIG. 14 is a flowchart illustrating a dispensing operation of the special dispensing mode in FIG. 5. FIG. 15 is a graph of pressure change when the dispensing probe 50 is lowered into and sucks out a specimen, which is separated into layers of blood plasma and blood cells. FIG. 16 is a diagram of a dispensing operation for dispensing blood plasma from a sample which is separated into layers of blood plasma and blood cells. FIG. 17 is a diagram of a dispensing operation for dispensing blood cells from a sample which is separated into layers of blood plasma and blood cells. FIG. 18 is a diagram of a washing operation after dispensation of blood cells from a sample which is separated into layers of blood plasma and blood cells.

As illustrated in FIG. 14, in the special dispensing mode (Step S103), it is first confirmed whether the sample to be dispensed is blood plasma or not (Step S300). Normally, the dispensing is performed from each of blood plasma in the upper layer and blood cells in the lower layer of a sample in which such blood plasma and blood cells are separated from one another in layers. However, since there is also a case where specimen dispensing is performed from either of them for reexamination or the like, the dispensing sample is confirmed. If the dispensing sample is blood plasma (Step S300: Yes), then under the control of the washing control section 200, the dispensing probe 50 is lowered into the specimen container 22 (Step S301). If the dispensing sample is not blood plasma (step S300: No), then the process moves to Step S308. The dispensing probe 50 continues to be lowered until the liquid level detecting section 51 detects a liquid level (Step S302: No). If a liquid level is detected (Step s302: Yes), then the lowering of the dispensing probe 50 is stopped. Subsequently, based on a blood plasma suction position, specimen container information and a blood plasma liquid level position, the calculating section 202 calculates a blood plasma liquid surface level, a blood plasma suction level and a contamination adhering range (Step S303). The blood plasma liquid surface level is a blood plasma liquid surface level within the specimen container 22, which is calculated from the specimen container information and the blood plasma liquid surface level. The blood plasma suction level is a level at which blood plasma is sucked out within the specimen, which is calculated from the blood plasma suction position and blood plasma liquid surface level set with regard to blood plasma samples. The contamination adhering range is a range of specimen adhering to the external wall of the dispensing probe 50, which is calculated from the specimen liquid surface level and the specimen suction level.

The dispensing probe 50 is lowered a given distance from the blood plasma liquid level position to the blood plasma suction level (Step S304) to suck out the blood plasma sample (Step S305). The sucked blood plasma is discharged to the reaction container 32 (Step S306), and the dispensing probe 50 is conveyed to the dispensing probe washing apparatus 6 (Step S307). The washing control section 200 controls the lowering speed of the dispensing probe 50 into the dispensing probe washing apparatus 6, to be a high speed, and also controls the insertion length of the dispensing probe 50 into the dispensing probe washing apparatus 6, based on the contamination adhering area calculated by the calculating section 202. The washing water controlling section 203 controls the supply amount of the washing water from the spout washing water supplying section 61 and the reserve washing water supplying section 63 in the dispensing probe washing apparatus 6 as well as the overflow amount from the reservoir vessel 62 to the overflow vessel 64 and the overflow timing.

After the washing of the dispensing probe 50, it is confirmed whether the sample to be dispensed is blood cells or not (Step S308). If the dispensing sample is blood cells (Step S308: Yes), then under the control of the washing control section 200, the dispensing probe 50 is lowered into and suck out the specimen, to detect the pressure change (Step S309). While the dispensing probe 50 is allowed to be lowered into the specimen, the sample is sucked out using the dispensing pump 55, and the pressure during the suction is detected by the pressure sensor 52. FIG. 15 is a graph of pressure change when the dispensing probe 50 is lowered into and sucks out a specimen, which is separated into layers of blood plasma and blood cells. When the dispensing probe 50 is lowered to suck out the specimen, the suction pressure changes at the liquid level of the blood plasma (t1-t2). When dispensing probe 50 sucks out the blood plasma and is lowered (t2-t3), the pressure is constant (V1). When the dispensing probe 50 is further lowered for suction, the pressure changes around the blood plasma-blood cells interface (t3-t4). When the dispensing probe 50 is completely inserted in the blood cell sample layer (blood plasma-blood cells interface), the suction pressure again becomes constant (V2). Therefore, the lowering and suction of the dispensing probe 50 are stopped after the interface detecting section 201 recognizes the blood plasma-blood cells interface (t5). In addition, in FIG. 15, as the dispensing probe 20b sucks out the specimen and is lowered, the suction pressure changes at the point when the dispensing probe 20b sucks out the blood plasma sample (t1). Therefore, it is also possible to detect a blood plasma liquid level by detecting the subject pressure change.

The dispensing probe sucks out the specimen and is lowered until the interface detecting section 201 detects the interface (Step S310: No). After the detection of the interface (Step s310: Yes), the lowering of the dispensing probe 50 is stopped. Subsequently, based on a blood cell suction position, specimen container information and a blood cell liquid surface level (interface position), the calculating section 202 calculates a blood cell liquid surface level, a blood cell suction level and a contamination adhering range of each layer (Step S311). The blood cell liquid surface level is a blood cell liquid surface level within the specimen container 22, which is calculated from the specimen container information and the blood cell liquid surface level (interface position). The blood cell suction level is a level at which the blood cells are sucked out within the specimen, which is calculated from a blood cell suction position, a blood cell dispensing amount and a blood cell liquid surface level, set with regard to blood cell samples. The contamination adhering portion from the specimen which is separated into layers includes a contamination portion due to the adhesion of blood cells and a contamination portion due to the adhesion of blood plasma. The contamination adhering range is calculated for each layer. The contamination adhering range for each layer is calculated from the blood plasma liquid surface level, the blood cell liquid surface level and the blood cell suction level, based on the insertion length of the dispensing probe 50 into the specimen which is separated into layers.

The dispensing probe 50 is lowered a given distance from the blood cell liquid surface position to the blood cell suction level (Step S312), the blood cell sample is sucked out (Step S313), and the sucked blood cells are discharged to the reaction container 32 (Step S314). Subsequently, based on the sample type information of the specimen obtained at Step S101 as well as the contamination adhering area of each component calculated at Step S303 and Step S311, the washing control section 200 and the washing water controlling section 203 set the washing range, lowering speed and/or washing water amount of the dispensing probe 50 in the dispensing probe washing apparatus 6 (Step S315). After the dispensing probe 50 is conveyed to the dispensing probe washing apparatus 6, the dispensing probe 50 is washed under the washing conditions set at Step S315 (Step S316). The washing control section 200 first controls the lowering speed of the dispensing probe 50 into the dispensing probe washing apparatus 6 to be a low speed, to wash the blood cells adhered to the tip portion of the dispensing probe 50. After the washing of the blood cell portion, the washing control section 200 controls the lowering speed of the dispensing probe 50 into the dispensing probe washing apparatus 6 to be switched to a high speed, to wash the blood plasma adhered to the upper part of the dispensing probe 50. The washing control section 200 also controls the insertion length of the dispensing probe 50 into the dispensing probe washing apparatus 6, based on the contamination adhering area calculated by the calculating section 202. The washing water controlling section 203 controls the supply amount of the washing water from the spout washing water supplying section 61 and the reserve washing water supplying section 63 in the dispensing probe washing apparatus 6 as well as the overflow amount from the reservoir vessel 62 to the over flow vessel 64 and the overflow timing. In addition, when the type of sample that is difficult to wash, such as blood cells, is washed off, the washing water controlling section 203 may control the supply amount of the washing water L2 to be increased from the nozzle section 61a of the spout washing water supplying section 61 and/or the nozzle section 63a of the reserve washing water supplying section 63.

In the blood plasma dispensing, as illustrated in FIG. 16(a), the dispensing probe 50 is lowered into the specimen container 22. When the liquid level detecting section 51 detects a liquid level, the lowering of the dispensing probe 50 is paused. Based on the detected blood plasma liquid level position, a set blood plasma suction position, and specimen container information, the calculating section 202 calculates a blood plasma liquid surface level h₅ and a blood plasma suction level h₆. As illustrated in FIG. 16(b), under the control of the washing control section 200, the dispensing probe 50 is lowered a given distance from the blood plasma liquid level position to the blood plasma suction level h₆, and the blood plasma specimen BP is sucked out. The sucked blood plasma specimen BP is discharged to the reaction container 32 (see FIG. 16(c)). The blood plasma BP corresponding to the length of the dispensing probe 50 inserted into the specimen is adhered to the external wall surface of the dispensing probe 50 after the discharge of the blood plasma, and the calculating section 202 calculates an area of a contamination adhering range S₃ based on the blood plasma liquid surface level h₅ and the blood plasma suction level h₆ (see FIG. 16(d)). The dispensing probe 50 after the dispensing of the blood plasma specimen is washed in the same manner as the dispensing probe 50 after dispensing the blood plasma sample which is not separated into layers; however, the subject washing may be omitted.

In the blood plasma dispensing, as illustrated in FIG. 17(a), the dispensing probe 50 is lowered for suction into the specimen container 22 to detect the pressure change. When the interface detecting section 201 detects an interface, the lowering of and sucking out by the dispensing probe 50 is paused. Based on the detected the blood cell liquid level position (interface position), a set blood cell suction position and specimen container information, the calculating section 202 calculates a blood cell liquid surface level h₇ and a blood cell suction level h₈. As illustrated in FIG. 17(b), under the control of the washing control section 200, the dispensing probe 50 is lowered a given distance from the blood cell liquid level position to the blood cell suction level h₈, and the blood cell specimen BC is sucked out. The sucked blood cell specimen BC is discharged to the reaction container 32 (see FIG. 16(c)). The blood plasma BP and blood cells BC corresponding to the length of the dispensing probe 50 inserted into the specimen are adhered to the external wall surface of the dispensing probe 50 after the discharge of the blood cells, and the calculating section 202 calculates an area of a blood cell contamination adhering range S₄ and a blood plasma contamination adhering range S₅ based on the blood plasma liquid surface level h₅ calculated at the blood plasma suction, blood cell liquid surface level h₇ and blood cell suction level h₈ (see FIG. 17(d)).

After the blood cell specimen BC is dispensed to the reaction container 32, as illustrated in FIG. 18(a), the dispensing probe 50 is conveyed to the dispensing probe washing apparatus 6, and the discharging of the specimen remaining inside the dispensing probe 50 and the washing of the internal wall surface thereof are first performed. By the driving of the pump driving section 56 and the moving of the piston 55a in an advanced manner, the remaining specimen is discharged from the dispensing probe 50 together with the extrusion liquid L1. At this stage, the reservoir vessel 62 is controlled by the washing water controlling section 203 such that at least the washing water L2 is supplied from the reserve washing water supplying section 63 before the extrusion liquid L1 falls into and reaches the reservoir vessel 62 by the advancement of the piston 55a, to be allowed to overflow along the inclined surface formed between the reservoir vessel 62 and the overflow vessel 64, from the opening 62a of the reservoir vessel 62 to the overflow vessel 64. When the extrusion liquid L1 is falling into the reservoir vessel 62, it is controlled to overflow from the reservoir vessel 62 to the overflow vessel 64. By this overflow, the discharged extrusion liquid L1 together with the remaining specimen is discharged to the overflow vessel 64 together with the overflow washing water L2, thus maintaining the degree of cleanliness of the washing water L2 in the reservoir vessel 62.

With the stop of the discharging of the extrusion liquid L1, the washing of the internal wall surface of the dispensing probe 50 ends, and the washing of the external wall surface of the dispensing probe 50 is then performed. The blood cell contamination part S₄ and the blood plasma contamination part S₅ are adhered to the external wall surface of the dispensing probe 50, after the dispensing of the blood cell specimen, in accordance with the insertion length into the specimen which is separated into layers. Since the blood cell specimen is most difficult to wash, the washing control section 200 sets the lowering speed of the blood cell contamination part S₄ of the dispensing probe 50 into the washing vessel 60, to be the lowest. The dispensing probe 50 is allowed to be lowered and advance, at a low speed, in the flow path created by spouting of the washing water L2 from the nozzle section 61a of the spout washing water supplying section 61, at an upper part of the reservoir vessel 62 from which the washing water L2 is allowed to overflow, and the blood cells adhered to the external wall surface of the dispensing probe 50 are washed (see FIG. 18(b)). In a state where the washing water L2 is supplied from the nozzle section 63a of the reserve washing water supplying section 63 and the washing water L2 is allowed to overflow from the opening 62a of the reservoir vessel 62 into the overflow vessel 64, the dispensing probe 50 is allowed to be lowered and advance, at a medium speed, to be inserted into the opening 60a of the washing vessel 60. The washing water L2 injected out of the nozzle section 61a hits the external wall surface of the dispensing probe 50 along the longitudinal direction (advancing direction) of the dispensing probe 50, and the blood cell specimen adhered to the external wall surface of the dispensing probe 50 is removed, thus washing the external wall surface of the dispensing probe 50. Although the removed specimen falls into the reservoir vessel 62 together with the washing water L2, the reservoir vessel 62 is allowed to overflow the washing water L2 from the opening 62a. Thus, the washing water L2 injected from the nozzle section 61a is discharged to the overflow vessel 64 together with the overflow washing water L2 and the degree of cleanliness of the washing water L2 within the reservoir vessel 62 is maintained.

When the dispensing probe 50 is lowered, the washing water controlling section 203 performs control such that the washing water L2 is spouted from the nozzle section 61a of the spout washing water supplying section 61, from the time at which the tip section of the dispensing probe is expected to advance into the washing water L2 being spouted until the time at which the upper part of the external wall surface above the blood plasma contamination adhering part S₂ portion of the dispensing probe 50 is expected to advance into the washing water L2 being spouted and a certain period of time elapses. Further, during the time which the washing water L2 from the nozzle section 61a falls into the reservoir vessel 62, the washing water controlling section 203 performs control such that the washing water L2 is supplied from the reserve washing water supplying section 63 and the washing water L2 overflows to the overflow vessel 64.

During the washing with the injection of the washing water L2 from the nozzle section 61a of the spout washing water supplying section 61, under the control of the washing control section 200, while the dispensing probe 50 is lowered at a low speed, the dispensing probe 50 is inserted and dipped in the reservoir vessel 62, from which the washing water L2 is overflows to the overflow vessel 64 by the supply of the washing water L2 from the reserve washing water supplying section 63 (see FIG. 18(c)). After the blood cell contamination adhering range S₄ on the external wall surface of the dispensing probe 50 is completely inserted and dipped in the washing water L2 within the reservoir vessel 62 (blood cell contamination adhering range S₄ plus some extra), the washing control section 200 controls the lowering speed of the dispensing probe 50 to be switched to a high speed, and stops the lowering after the blood plasma contamination adhering range S₅ is completely inserted and dipped in the washing water L2 in the reservoir vessel 62. The washing water controlling section 203 performs control such that the washing water L2 is supplied from the reserve washing water supplying section 63 and the washing water L2 overflows to the overflow vessel 64 until the blood plasma contamination adhering range S₅ portion of the dispensing probe 50 is completely lowered and dipped in the washing water L2 reserved in the reservoir vessel 62.

After the blood plasma contamination adhering range S₅ of the dispensing probe 50 is completely dipped in the washing water L2 in the reservoir vessel 62, the washing water controlling section 203 stops supplying the washing water L2 from the reserve washing water supplying section 63 to the reservoir vessel 62, and the washing control section 200 allows the dispensing probe 50 to ascend and to be pulled out of the washing water L2 reserved in the reservoir vessel 62 (see FIG. 18(d)). During the pulling up of the dispensing probe 50, the overflow from the reservoir vessel 62 is stopped, and the pulling speed is controlled, so that the amount of the washing water L2 adhered to the dispensing probe 50 can be reduced. After the pulling up of the dispensing probe 50 from the washing water L2 in the reservoir vessel 62, the electromagnetic valve 62d is operated to be opened, so that the washing water L2 in the reservoir vessel 62 is discharged into the disposal tank 62c, and the washing ends (see FIG. 18(e)). In the Embodiments of the present invention, the lowering speed and insertion length of the dispensing probe 50 to the dispensing probe washing apparatus 6 can be controlled for washing in accordance with adhered contamination, specifically, the type of sample to be sucked out and the contamination adhering area, making it possible to reduce the washing time and the amount of the washing water to be used.

Further, in the Embodiments of the present invention, while the lowering speeds of the dispensing probe 50 to the dispensing probe washing apparatus 6 are controlled to be switched in accordance with the type of sample, the lowering speeds may also be controlled in more details in accordance with the viscosity of the samples. For example, it is confirmed that the blood of diabetics has higher viscosity than that of healthy people and such blood is more difficult to wash off. Accordingly, if samples are from diabetics, the lowering speed of the dispensing probe 50 to the dispensing probe washing apparatus 6 can be controlled to be slower than the case with healthy people to perform the washing. As a result, washing defects can be prevented from occurring.

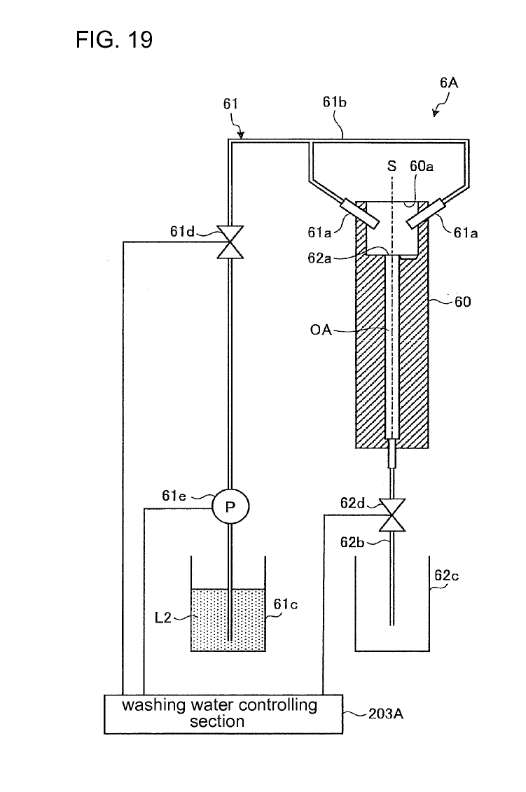

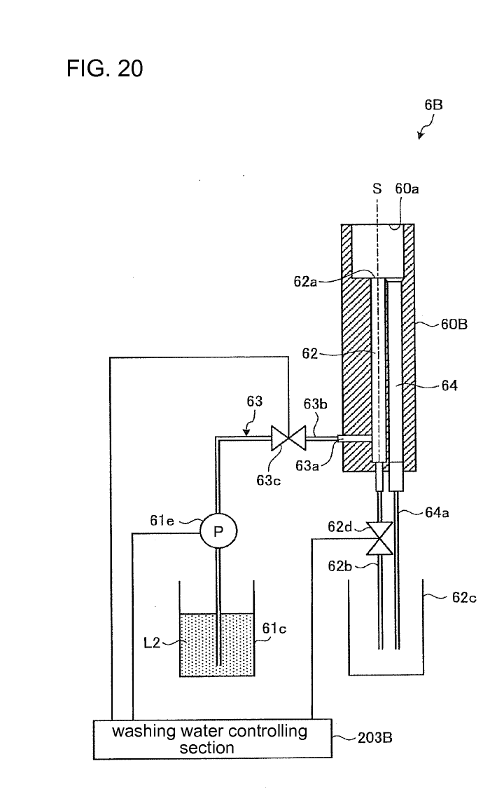

It is preferable for the dispensing probe washing apparatus used for the analyzer 1 according to the present invention to be the dispensing probe washing apparatus 6, which has a structure capable of allowing overflow from the reservoir vessel 62 to the overflow vessel 64 and capable of spout-washing by the spout washing water supplying section 61; however, when a high level analysis accuracy is not required depending on specimens to be analyzed or an analysis menu, dispensing probe washing apparatuses 6A and 6B as illustrated in FIGS. 19 and 20 can also be used. The dispensing probe washing apparatus 6A illustrated in FIG. 19 comprises only the spout washing water supplying section 61, and performs spout-washing on the dispensing probe 50. The dispensing probe washing apparatus 6B illustrated in FIG. 20 has a structure capable of allowing overflow from the reservoir vessel 62 to the overflow vessel 64, and dip-washes the dispensing probe 50 while performing overflow. In a case where the washing is performed also by the dispensing probe washing apparatuses 6A and 6B as illustrated in FIGS. 19 and 20, the washing control section 200 and the washing water controlling section 203 perform washing while controlling the lowering speed of the dispensing probe 50 to the dispensing probe washing apparatuses 6A and 6B, the washing range and/or the amount of the washing water, in accordance with dispensed samples, thereby reducing the washing time and the amount of washing water to be used.

Further, as a variation example of the Embodiments of the present invention, a dispensing apparatus comprising a liquid level estimating section for estimating a liquid level and/or interface of a specimen to be dispensed, is illustrated. In the analyzer according to the variation example, the storage section 104 stores a maximum housed specimen amount for each specimen container type, in addition to analysis conditions, such as the sample type of specimens, and specimen container shapes corresponding to specimen container types. In the variation example, a specimen container ID is read by the reading section 23, and based on the read specimen container ID, the shape of the specimen container and the maximum specimen amount housed in the specimen container are extracted. The liquid level estimating section estimates a liquid level and/or an interface based on the extracted specimen container shape and the maximum housed specimen amount. When dispensation is performed with one specimen for a plurality of different analyses in an analysis menu, the liquid level and/or the interface are estimated in consideration of the dispensing amount thereof. Based on the estimated liquid level and/or interface, the sample type of the specimen, and the specimen suction position, the calculating section 203 calculates a contamination adhering range on the external wall surface of the dispensing probe 50. Although the accuracy of the liquid level and/or interface position estimated by the liquid level estimating section is less than the liquid level detected by the liquid level detecting section, the contamination adhering range can be readily calculated for different specimen container types.

### INDUSTRIAL APPLICABILITY

As described above, the analyzer and the method for washing a dispensing probe according to the present invention are useful for an analyzer capable of analyzing different specimen types by itself, and in particular, they are suitable for a case where the total washing time is shortened to perform analysis in an effective manner.

### DESCRIPTION OF THE REFERENCE NUMERALS

1 analyzer
2 specimen table
3 reaction table
4 reagent table
5 specimen dispensing apparatus
6, 6A, 6B, 8 dispensing probe washing apparatus
7 reagent dispensing apparatus
9 measurement mechanism
10 control mechanism
21, 31, 41 housing section
22 specimen container
22a, 42a opening
23, 43 reading section
32 reaction container
33 photometry apparatus
33a light source
33b light receiving section
34 reaction container washing apparatus
42 reagent container
50 dispensing probe
51 liquid level detecting section
52 pressure sensor
53 probe driving section
54 tube
55 dispensing pump
56 pump driving section
57 tank
58 electromagnetic valve
59 washing water pump
60 washing vessel
60a opening
61 spout washing water supplying section
61a nozzle section
61b, 62b, 63b, 64a tube
61c tank
61d, 62d, 63c electromagnetic valve
61e pump
62 reservoir vessel
62a opening
62c disposal tank
63 reserve washing water supplying section
63a nozzle section
64 overflow vessel
101 controlling section
102 input section
103 analysis section
104 storage section
105 output section
106 display section
107 transmission and reception section
200 washing control section
201 interface detecting section
202 calculating section
203, 203A, 203B washing water controlling section
L1 extrusion liquid
L2 washing water
BP blood plasma
WB whole blood
BC blood cells
S center line

1. An analyzer capable of analyzing different samples as a specimen, the analyzer optically analyzing a reactant of the specimen and a reagent, characterized in comprising:
an analysis information obtaining section for storing or obtaining specimen information including a sample type of the specimen and a specimen suction position, and specimen container information;
a liquid level estimating section for estimating a liquid level and/or an interface of the specimen;
a dispensing section for dispensing the specimen using a dispensing probe;
a washing section for washing the dispensing probe;
a calculating section for calculating a contamination adhering range of an external wall surface of the dispensing probe, based on the sample type, the specimen suction position, and the specimen container information stored or obtained by the analysis information obtaining section as well as liquid level and/or interface information of the specimen estimated by the liquid level estimating section; and
a washing control section for controlling a washing range of the dispensing probe, based on the contamination adhering range of the external wall surface of the dispensing probe calculated by the calculating section.

2. An analyzer capable of analyzing different samples as a specimen, the analyzer optically analyzing a reactant of the specimen and a reagent, characterized in comprising:
an analysis information obtaining section for storing or obtaining specimen information including a sample type of the specimen and a specimen suction position;
a liquid level detecting section for detecting a liquid level and/or an interface of the specimen;
a dispensing section for dispensing the specimen using a dispensing probe;
a washing section for washing the dispensing probe;
a calculating section for calculating a contamination adhering range of an external wall surface of the dispensing probe, based on the sample type, the specimen suction position, and the specimen container information stored or obtained by the analysis information obtaining section as well as liquid level and/or interface information of the specimen detected by the liquid level detecting section; and
a washing control section for controlling a washing range of the dispensing probe, based on the contamination adhering range of the external wall surface of the dispensing probe calculated by the calculating section.

3. The analyzer according to claim 1, characterized in that the washing control section controls a lowering speed of the dispensing probe into the washing section based on the sample type of the specimen.

4. The analyzer according to claim 1, characterized in that the specimen information includes a sample type, sample viscosity, a specimen dispensing amount, a specimen suction position and an analysis menu information.

5. The analyzer according to claim 1, characterized in that the washing control section controls the lowering speed of the dispensing probe in accordance with a viscosity category of the specimen.

6. The analyzer according to claim 1, characterized in that the washing section comprises:
a washing vessel having an opening at an upper part thereof, the opening allowing the dispensing probe to be inserted therein;
a spout washing water supplying section for spouting washing water in a region of the upper part of the washing vessel; and
a washing water controlling section for controlling the amount of the washing water spouted from the spout washing water supplying section based on the sample type of the specimen to be washed.

7. The analyzer according to claim 1, characterized in that the washing section comprises:
a reservoir vessel for allowing overflow the washing water, the reservoir vessel having an opening at an upper part thereof for allowing the dispensing probe to be inserted therein;
an overflow vessel for discharging the overflow washing water from the reservoir vessel;
a reserve washing water supplying section for supplying the washing water to the reservoir vessel; and
a washing water controlling section for controlling the amount of the washing water supplied from the reserve washing water supplying section based on the sample type of the specimen to be washed.

8. The analyzer according to claim 7, characterized in that the washing water controlling section controls at least the reservoir vessel to be in an overflow state before an extrusion liquid, which is discharged from the dispensing probe to wash an internal wall surface thereof at an upper part of the reservoir vessel, falls and reaches the washing water reserved in the reservoir vessel.

9. The analyzer according to claim 7, characterized in that the washing water controlling section controls at least the reservoir vessel to be in an overflow state before a tip of the dispensing probe is dipped in the washing water reserved in the reservoir vessel by the lowering of the dispensing probe into the washing section.

10. The analyzer according to claim 7, characterized in that an opening of the overflow vessel is formed to have an inclined surface, which inclines downwardly from the opening of the reservoir vessel.

11. The analyzer according to claim 9, characterized in that the washing water controlling section performs a control to allow the washing water reserved in the reservoir vessel to overflow before the extrusion liquid discharged from the dispensing probe for washing the internal wall surface thereof falls into and reach the washing water, and after the discharging of the extrusion liquid for washing the internal wall surface from the dispensing probe ends, the washing water controlling section performs a control to allow the washing water reserved in the reservoir vessel to stop overflowing after the discharged extrusion liquid falls and reaches the washing water.

12. The analyzer according to claim 9, characterized in that the washing water controlling section performs a control to resume the overflow before the dispensing probe is lowered in the washing section and the tip of the dispensing probe is dipped in the washing water reserved in the reservoir vessel, and the washing water controlling section performs a control to stop the overflow before the dispensing probe is pulled up from the reservoir vessel.

13. The analyzer according to claim 7, characterized in that the analyzer comprises a spout washing water supplying section for spouting washing water in an upper part region of the washing vessel.

14. The analyzer according to claim 13, characterized in that the washing water controlling section controls the washing water spouted from the spout washing water supplying section to allow at least the reservoir vessel to be in an overflow state when the washing water falls into the reservoir vessel.

15. The analyzer according to claim 13, characterized in that the washing control section selects, for performing washing, washing by washing-water spouting by the spout washing water supplying section and/or dip-washing with the washing water reserved in the reservoir vessel, in accordance with the sample type dispensed by the dispensing probe.

16. The analyzer according to claim 7, characterized in that the calculating section calculates a specimen contamination range of each layer of the external wall surface of the dispensing probe, based on a suction position of each layer of the specimen which is separated into layers, and the specimen container information, stored or obtained by the analysis information obtaining section, as well as liquid level position information of each layer of the specimen which is separated into layers, detected by the liquid level detecting section; the washing control section controls an insertion distance or the lowering speed of the dispensing probe to the washing section in accordance with the specimen contamination range of each layer calculated by the calculating section; and/or the washing water controlling section controls the amount of the washing water.

17. The analyzer according to claim 2, characterized in that the liquid level detecting section comprises:
a liquid level detecting part for detecting a liquid level from the change in capacitance between the dispensing probe and a metal plate disposed in the periphery of a specimen container; and
an interface detecting section for detecting a pressure during ascent or descent while performing suction of the dispensing probe in the specimen using a pressure detecting section within the dispensing probe, to detect an interface of the specimen which is separated into layers in a specimen container, based on the detected pressure change.

18. The analyzer according to claim 1, characterized in that the sample type of the specimen is blood plasma, blood serum, whole blood or a specimen which is separated into layers of blood plasma and blood cells.

19. A method for washing a dispensing probe after dispensation of a specimen, characterized in comprising:
a reading step of reading a specimen ID of a specimen to be dispensed and a specimen container ID;
an extracting step of extracting analysis menu information including a specimen sample type and a specimen suction position, and specimen container information;
a liquid level detecting step of detecting a specimen liquid level;
a calculating step of calculating a specimen liquid surface level, a specimen suction level and a contamination adhering range of an external wall surface of a dispensing probe, based on a specimen liquid level position detected at the liquid level detecting step, the sample type, the specimen container information, and the specimen suction position;
a sucking step of sucking out a specimen at the specimen suction level;
a discharging step of discharging the sucked specimen to a reaction container; and
a washing step of washing the dispensing probe by controlling the insertion distance and the lowering speed of the dispensing probe to the washing section and/or the amount of the washing water, based on the sample type of the specimen and the contamination adhering range of the external wall surface of the dispensing probe.

20. The method for washing a dispensing probe according to claim 19, characterized in that the method comprises an internal wall surface washing step of performing internal wall surface washing by the discharging of an extrusion liquid from the dispensing probe, within the washing section, prior to the washing step.

21. A method for washing a dispensing probe after dispensation of a lower layer component of a specimen which is separated into layers, characterized in comprising:
a reading step of reading a specimen ID of a specimen to be dispensed and a specimen container ID;
an extracting step of extracting analysis menu information including a specimen sample type and a specimen suction position for each layer, and specimen container information;
a detecting step of detecting a specimen liquid level and an interface;
a calculating step of calculating a specimen liquid surface level of each layer, a specimen suction level of each layer and a contamination adhering range of each layer of an external wall surface of a dispensing probe, based on a specimen liquid level position and an interface position detected at the detecting step, the sample type, the specimen container information, and the specimen suction position of each layer;
a sucking step of sucking out a lower layer specimen at a lower layer specimen suction level;
a discharging step of discharging the sucked specimen to a reaction container;
a first washing step of washing the dispensing probe by controlling the insertion distance and the lowering speed of the dispensing probe to the washing section and/or the amount of the washing water, based on the sample type of the lower layer specimen and the contamination adhering range of the lower layer of the external wall surface of the dispensing probe; and
a second washing step of washing the dispensing probe by controlling the insertion distance and the lowering speed of the dispensing probe to the washing section and/or the amount of the washing water, based on the sample type of the upper layer specimen and the contamination adhering range of the upper layer of the external wall surface of the dispensing probe.

22. The method for washing a dispensing probe according to claim 21, characterized in that the method comprises an internal wall surface washing step of performing internal wall surface washing by the discharging of an extrusion liquid from the dispensing probe, within the washing section, prior to the first washing step.
